(19) United States
(12) Patent Application Publication
(10) Pub. No.: US 2022/0357316 A1
(43) Pub. Date: Nov. 10, 2022

(54) SYSTEM AND METHOD FOR CONDITIONING GAS FOR ANALYSIS

(71) Applicant: Biometry Inc., Boston, MA (US)

(72) Inventors: Bryan NOLAN, Brookline, MA (US); Thomas T. MORGAN, Stow, MA (US); Devon C. Campbell, Lexington, MA (US)

(21) Appl. No.: 17/425,635

(22) PCT Filed: Jan. 27, 2020
(86) PCT No.: PCT/US2020/015250

§ 371 Date: Jul. 23, 2021

Related U.S. Application Data
(60) Provisional application No. 62/797,147, filed on Jan. 25, 2019.

(51) Int. Cl. G01N 33/52; G01N 1/22; G01N 33/497

(52) U.S. Cl. CPC G01N 33/525; G01N 1/22; G01N 33/497; G01N 2001/2244

(57) ABSTRACT

Methods and systems for conditioning a gas sample for analysis and measuring, detecting, and/or determining the concentration of at least one analyte in a gas sample. Methods include a combination and/or repetition of dehumidifying and/or humidifying the gas, and/or performing a chemical reaction an analyte, and measuring, detecting, and/or determining the concentration of an analyte or an output analyte resulting from the chemical reaction. Systems to adjust the humidity of a gas sample and/or perform a chemical reaction on an analyte, and measure, detect, and/or determine the concentration of an analyte or an output analyte resulting from the chemical reaction comprise cartridges, capsules, test strips or test strip chambers and one or more sensors. Systems may further comprise a humidity exchange material to further adjust the humidity. Gas samples include exhaled breath. Analytes include nitric oxide. Output analytes include nitrogen dioxide.

### CROSS-REFERENCE TO RELATED APPLICATIONS

This application claims benefit under 35 U.S.C. § 119(e) to U.S. Provisional Patent Application No. 62/797,147, entitled System and Method for Conditioning Gas for Analysis, filed Jan. 25, 2019, the contents of which are hereby incorporated by reference in their entirety.

### INCORPORATION BY REFERENCE

All patents, patent applications, and publications cited herein are hereby incorporated by reference in their entirety in order to more fully describe the state of the art as known to those skilled therein as of the date of the invention described herein. International Patent Application Number PCT/US2015/000180, entitled Mini Point of Care Gas Chromatographic Test Strip and Method to Measure Analytes, filed Dec. 23, 2015, International Patent Application Number PCT/US2015/034869, entitled Low Cost Test Strip and Method to Measure Analyte, filed Jun. 9, 2015, International Patent Application Number PCT/US2017/042830, entitled Methods of and Systems for Measuring Analytes Using Batch Calibratable Test Strips, filed Jul. 19, 2017, which claims priority under 35 U.S.C. § 119(e) to U.S. Provisional Application No. 62/363,971, filed Jul. 19, 2016, entitled Methods of and Systems for Test Strip Regeneration and Sample Manipulation for Use With Same, which are hereby incorporated by reference in their entirety.

### BACKGROUND

### Field of Invention

This technology generally relates to systems and methods for conditioning gas for analysis and detecting and/or measuring at least one analyte in a gas sample. More specifically, the technology relates to systems and methods for conditioning gas and determining the concentration of an analyte.

### Context of the Technology

There are many different types of sensors and technologies available for gas and analyte detection known in the art. The problems associated with these sensors and detection systems have been discussed in the related applications incorporated above. Some of those shortcomings include cost, complexity, calibration, quality control, shelf life, ease of use, etc. This is not intended to be an exhaustive list.

One of the shortcomings of existing gas sensors is the cost and complexity of pre-conditioning gases to an acceptable humidity range. Existing sensors and sensing technologies are not capable of performing accurate measurements under high humidity or dynamic humidity conditions and therefore the sample must be pre-conditioned in order to perform an accurate measurement. Analyzing gases in breath provides an additional challenge in that breath exits the mouth with a relative humidity of 100% and a temperature of 37° C.

Pre-conditioning of the analyte for analysis can be performed with tubing made from humidity exchange materials such as perfluorosulfonic acids, perfluorocarboxylic acids, and polymers and co-polymers made there of (e.g. Nafion®). Nafion® tubing enables the sample to be dehumidified (e.g., in the case of breath), humidified (e.g., in the case of industrial gases purchased from a vendor such as Air Liquide), or to equilibrate humidity with the ambient conditions, without affecting the concentration of certain analytes. The efficiency of the Nafion® tube to humidify or dehumidify is dependent upon its length, diameter, and the flow rate of the gas. The higher the flow rate, the longer and larger diameter the Nafion® tube must be to equilibrate the sample with ambient conditions. This has a disadvantage because the longer the tube and wider the diameter of the Nafion® tube, the higher its cost. It is also limited by flow rate and therefore impacts the volume required by the sensor to perform an analysis.

Other desiccants and humectants have similar disadvantages. Their performance is based on the volume and surface area of desiccant/humectant material available to adsorb or desorb humidity from the sample. Their efficiency is impacted by the ambient conditions. A single use, or limited use, desiccants or humectants will adsorb, or desorb (respectively), a fixed amount of humidity each time it is exposed to the sample. For example, given a patient breath sample is saturated with 100% humidity, a desiccant may remove 40% of the humidity to reduce the sample to 60% relative humidity (RH). If the ambient humidity is 35% RH, there is a 25% delta between the relative humidity of the sample and ambient conditions, thus interfering with the ability of the sensor to perform an analysis. Desiccant materials are also at a disadvantage because they are only able to lower the humidity, not equilibrate it to ambient conditions. In the case where the ambient humidity is high, the desiccant may lower the sample humidity to be lower than ambient, resulting in a large fluctuation in the humidity that passes over the sensor. Alternatively, the use of dynamic chemical moisture stabilizers, or equilibrium stabilizers falls (e.g. combined humectants/desiccant sorbent packets, clay composites, or salt/cellulose composites, such as under the trade name Propadyn®) under similar constraints of volume and surface area, as well a single or limited use, and fixed humidity range, requiring that the stabilizers are “tuned” to a particular relative humidity, which may not match the ambient humidity on a given day

To address these problems, the disclosed technology conditions an incoming gas stream in order to deliver a more appropriate sample to a sensor or detector for analysis. One example of a single use, disposable sensor and re-usable measurement system has been previously described by International Patent Application Numbers PCT/US2015/000180, PCT/US2015/034869, and PCT/US2017/042830, hereby incorporated by reference in their entirety. Conditioning the gas stream may include but is not limited to, altering at least one of humidity, temperature, and/or pressure. Conditioning may also involve chemically altering at least one analyte in the sample. Examples of humidity modification includes but is not limited to dehumidifying, humidifying, or equilibrating the sample with ambient conditions, or combinations thereof.

### BRIEF SUMMARY OF THE INVENTION

In one embodiment, the technology is a system comprising:

a test strip comprising: one or more flexible layers defining one or more flexible layer holes, and one or more of a permanganate salt, silica, permanganate salt on silica, or activated carbon disposed in the one or more flexible layer holes; and
a tube in fluid communication with the test strip, wherein the tube comprises one or more of a perfluorosulfonic acid or a polymer or copolymer derived therefrom, a perflurocarboxylic acid or a polymer or copolymer derived therefrom, or a humidity exchange material; and one or more sensors to detect and/or measure an analyte.

In some aspects, the permanganate salt on silica is deposited in the one or more flexible layer holes. In some aspects, the permanganate salt on silica is a potassium permanganate.

In some aspects, the one or more flexible layer holes is tapered. In some aspects, the one or more flexible layer holes is circular, oval-shaped, square-shaped, or rectangular.

In some aspects, the system further comprises one or more membrane layers. In some aspects, the one or more membrane layers comprise a first membrane layer, and a second membrane layer; wherein the one or more flexible layers comprises a first flexible layer, wherein the first flexible layer has a first upper surface, wherein the first flexible layer has a first lower surface, and wherein the first flexible layer defines a first hole traversing the first upper surface and the first lower surface, wherein the first membrane is configured to overlay the first hole defined by the first upper surface of the first flexible layer, and wherein the second membrane layer has a first second-membrane surface, wherein the second membrane layer has a second second-membrane surface, and wherein the first second-membrane surface is configured to overlay the first hole defined by the first lower surface of the first flexible layer. In some aspects, the one or more of the permanganate salt, the silica, the permanganate salt on silica, or the activated carbon is deposited in the first hole. In some aspects, the permanganate salt on silica is deposited in the first hole. In some aspects, the permanganate salt on silica is a potassium permanganate.

In some aspects, the one or more flexible layers further comprises a second flexible layer, the one or more membrane layers further comprises a third membrane layer, the second flexible layer has a second upper surface, wherein the second flexible layer has a second lower surface, and wherein the second flexible layer defines a second hole traversing the second upper surface and the second lower surface, the second membrane layer is disposed between the first flexible layer and the second flexible layer, and the third membrane layer is configured to overlay the second hole defined by the second lower surface of the second flexible layer.

In some aspects, the one or more membrane layers further comprises a fourth membrane layer, the fourth membrane layer has a first fourth-membrane surface, the fourth membrane layer has a second fourth-membrane surface, the fourth membrane is disposed between the second membrane layer and the second flexible layer, and the second fourth-membrane surface is configured to overlay the second hole defined by the second upper surface of the second flexible layer.

In some aspects, the total number of the one or more flexible layers is n, the total number of the one or more membranes is m, and m is equal to n, n+1, or n−1.

In some aspects, the one or more of the permanganate salt, the silica, the permanganate salt on silica, or the activated carbon is deposited in the second hole. In some aspects, the permanganate salt on silica is deposited in the second hole. In some aspects, the permanganate salt on silica deposited in the second hole is a potassium permanganate.

In some aspects, the system further comprises one or more protective layers, wherein the one or more protective layers comprises a first protective layer configured to overlay the second surface of the first membrane layer. In some aspects, the first protective layer defines a protective layer hole. In some aspects, the protective layer hole defined by the first protective layer is configured to provide fluid communication between the one or more of the permanganate salt, the silica, the permanganate salt on silica, or the activated carbon of the test strip and the tube.

In some aspects, the sensor is a sensing layer. In some aspects, the test strip comprises the sensing layer. In some aspects, the sensing layer defines one or more sensing layer holes. In some aspects, the one or more sensing layer holes defined by the sensing layer is configured to provide fluid communication between the one or more of the permanganate salt, the silica, the permanganate salt on silica, or the activated carbon of the test strip and the tube. In some aspects, the sensing layer comprises one or more electrodes. In some aspects, the sensing layer comprises one or more sensing chemistries. In some aspects, the sensing layer further comprises one or more electrodes, and the one or more sensing chemistries is configured to bridge the one or more electrodes.

In some aspects, the test strip comprises one or more spacing layers, and the one or more spacing layers defines one or more spacing layer holes.

In some aspects, the system further comprises a housing, and the housing is configured to provide fluid communication between one or more of the test strip, the one or more sensors, and the tube. In some aspects, the housing is configured to provide fluid communication between the test strip and the tube. In some aspects, the system further comprises a pump, a blower, or a fan connected to the housing, wherein the pump, the blower, or the fan is configured advance a gas through the system.

In some aspects, the system further comprises one or more chamber layers at least in part defining a chamber, and the chamber comprises one or more of a chamber membrane, a chamber frit, or a chamber filter. In some aspects, the one or chamber layers comprises one or more protective layers, and/or one or spacing layers. In some aspects, the chamber comprises one or more of a permanganate salt, silica, a permanganate salt on silica, or an activated carbon. In some aspects, the chamber comprises the permanganate salt on silica. In some aspects, the chamber is tapered.

In some aspects, the system further comprises one or more of: a pressure sensitive adhesive; a heat sensitive adhesive; a sonic weld; a bond; a two-part adhesive; or a moisture-cure adhesive. In some aspects, the system further comprises one or more humectants. In some aspects, the one or more humectants comprises: polypropylene glycol; glycerin; sodium hexamethyl phosphate; a glycol; a sugar alcohol; or glyceryl triacetate. In some aspects, the system further comprises one or more desiccants. In some aspects, the one or more desiccants comprises: a silica gel; an activated alumina; a bentonite clay; calcium sulfate; magnesium sulfate; or sodium chloride. In some aspects, the system further comprises one or more humidity stabilizing materials. In some aspects, the one or more humidity stabilizing materials comprises: magnesium chloride; a hydroxylmethyl cellulose composites; a clay composite; a silica gel; or Propadyn.

In some aspects, the one or more sensors comprises a chemoreceptive sensor. In some aspects, the one or more sensors comprises a metal oxide sensor. In some aspects, the one or more sensors comprises an electrochemical sensor. In some aspects, the one or more sensors comprises a chemiresistive sensor.

In one embodiment the technology is a system comprising

a test strip comprising: one or more flexible layers defining one or more flexible layer holes, one or more of a permanganate salt, silica, permanganate salt on silica, or activated carbon disposed in the one or more flexible layer holes, and one or more spacing layers defining one or more channels; and one or more sensors to detect and/or measure an analyte, wherein the one or more channels are configured to provide fluid communication for a gas between the test strip and the one or more sensors.

In some aspects, the one or more channels provide fluid communication for the gas to the one or more sensors or sensing chemistries subsequent to the gas traversing the one or more of the permanganate salt, the silica, the permanganate salt on silica, or the activated carbon of the test strip. In some aspects, the permanganate salt on silica is deposited in the one or more flexible layer holes. In some aspects, the permanganate salt on silica is a potassium permanganate. In some aspects, the one or more flexible layer holes is tapered. In some aspects, the one or more flexible layer holes is circular, oval-shaped, square-shaped, or rectangular.

In some aspects, the system further comprises one or more membrane layers. In some aspects, the one or more membrane layers comprise a first membrane layer, and a second membrane layer; wherein the one or more flexible layers comprises a first flexible layer, wherein the first flexible layer has a first upper surface, wherein the first flexible layer has a first lower surface, and wherein the first flexible layer defines a first hole traversing the first upper surface and the first lower surface, wherein the first membrane is configured to overlay the first hole defined by the first upper surface of the first flexible layer, and wherein the second membrane layer has a first second-membrane surface, wherein the second membrane layer has a second second-membrane surface, and wherein the first second-membrane surface is configured to overlay the first hole defined by the first lower surface of the first flexible layer.

In some aspects, the one or more of the permanganate salt, the silica, the permanganate salt on silica, or the activated carbon is deposited in the first hole. In some aspects, the permanganate salt on silica is deposited in the first hole. In some aspects, the permanganate salt on silica is a potassium permanganate.

In some aspects, the one or more flexible layers further comprises a second flexible layer, the one or more membrane layers further comprises a third membrane layer, the second flexible layer has a second upper surface, wherein the second flexible layer has a second lower surface, and wherein the second flexible layer defines a second hole traversing the second upper surface and the second lower surface, the second membrane layer is disposed between the first flexible layer and the second flexible layer, and the third membrane layer is configured to overlay the second hole defined by the second lower surface of the second flexible layer.

In some aspects, the one or more membrane layers further comprises a fourth membrane layer, the fourth membrane layer has a first fourth-membrane surface, the fourth membrane layer has a second fourth-membrane surface, the fourth membrane is disposed between the second membrane layer and the second flexible layer, and the second fourth-membrane surface is configured to overlay the second hole defined by the second upper surface of the second flexible layer.

In some aspects, the total number of the one or more flexible layers is n, the total number of the one or more membranes is m, and m is equal to n+1.

In some aspects, the one or more of the permanganate salt, the silica, the permanganate salt on silica, or the activated carbon is deposited in the second hole. In some aspects, the permanganate salt on silica is deposited in the second hole. In some aspects, the permanganate salt on silica is a potassium permanganate.

In some aspects, the system further comprises one or more protective layers, wherein the one or more protective layers comprises a first protective layer configured to overlay the second first-membrane surface of the first membrane layer. In some aspects, the first protective layer defines a protective layer hole.

In some aspects, the sensor is a sensing layer. In some aspects, the test strip comprises the sensing layer. In some aspects, the sensing layer defines one or more sensing layer holes. In some aspects, the sensing layer comprises one or more electrodes. In some aspects, the sensing layer comprises one or more sensing chemistries. In some aspects, the sensing layer further comprises one or more electrodes, and the one or more sensing chemistries is configured to bridge the one or more electrodes.

In some aspects, the system further comprises one or more chamber layers at least in part defining a chamber, and the chamber comprises one or more of a chamber membrane, a chamber frit, or a chamber filter. In some aspects, the one or chamber layers comprises one or more protective layers, and/or one or spacing layers. In some aspects, the chamber comprises one or more of a permanganate salt, silica, a permanganate salt on silica, or an activated carbon. In some aspects, the chamber comprises the permanganate salt on silica. In some aspects, the chamber is tapered.

In some aspects, the system further comprises one or more of: a pressure sensitive adhesive; a heat sensitive adhesive; a sonic weld; a bond; a two-part adhesive; or a moisture-cure adhesive. In some aspects, the system further comprises one or more humectants. In some aspects, the one or more humectants comprises: polypropylene glycol; glycerin; sodium hexamethyl phosphate; a glycol; a sugar alcohol; or glyceryl triacetate. In some aspects, the system further comprises one or more desiccants. In some aspects, the one or more desiccants comprises: a silica gel; an activated alumina; a bentonite clay; calcium sulfate; magnesium sulfate; or sodium chloride. In some aspects, the system further comprises one or more humidity stabilizing materials. In some aspects, the one or more humidity stabilizing materials comprises: magnesium chloride; a hydroxylmethyl cellulose composites; a clay composite; a silica gel; or Propadyn.

In some aspects, the one or more sensors comprises a chemoreceptive sensor. In some aspects, the one or more sensors comprises a metal oxide sensor. In some aspects, the one or more sensors comprises an electrochemical sensor. In some aspects, the one or more sensors comprises a chemiresistive sensor.

In one embodiment the technology is a method of conditioning a gas sample, the gas sample having a humidity and comprising one or more input analytes, wherein the method comprises:

a. providing the gas sample to a gas sample receiver;
b. adjusting the humidity of the gas sample;
c. providing the gas sample to a tube comprising one or more of a perfluorosulfonic acid, a perflurocarboxylic acid, or a humidity exchange material; and
d. adjusting the humidity of the gas sample to conditions equal to or about equal to ambient humidity; and
e. detecting or measuring one or more readout analytes, wherein detecting or measuring the one or more readout analytes follows step (a) and step (b).

In some aspects, the gas sample receiver comprises one of a cartridge or a capsule, wherein the cartridge or the capsule comprises one or more of one or more membranes, one or more frits, or one or more filters, and the gas sample passes through the one or more of the one or more membranes, the one or more frits, or the one or more filters in step (a). In some aspects, the one or more membranes, one or more frits, or one or more filters comprises one or more of a humidity exchange material, a selective membrane, a size exclusion membrane, a particulate filter, or a porous polypropylene.

In some aspects, the gas sample receiver comprises a test strip, wherein the test strip comprises one or more of membranes, and the gas sample passes through the one or more membranes in step (a). In some aspects, the one or more membranes comprises one or more of a humidity exchange material, a selective membrane, a size-exclusion membrane, a particulate filter, or a porous polypropylene.

In some aspects, the cartridge or the capsule comprises one or more conditioning materials, and the gas sample passes through the one or more conditioning materials in step (a). In some aspects, the cartridge or the capsule comprises one or more humectants, and the gas sample passes through the one or more humectants in step (a). In some aspects, the cartridge or the capsule comprises one or more desiccants, and the gas sample passes through the one or more desiccants in step (a). In some aspects, the cartridge or the capsule comprises one or more humidity stabilizing materials.

In some aspects, the test strip comprises one or more conditioning materials, and the gas sample passes through the one or more conditioning materials in step (a). In some aspects, the test strip comprises one or more humectants, and wherein the gas sample passes through the one or more humectants in step (a). In some aspects, the test strip comprises one or more desiccants, and the gas sample passes through the one or more desiccants in step (a).

In some aspects, the cartridge or the capsule comprises one or more humidity stabilizing materials.

In some aspects, the one or more humectants comprises: polypropylene glycol; glycerin; sodium hexamethyl phosphate; a glycol; a sugar alcohol; or glyceryl triacetate. In some aspects, the one or more desiccants comprises: a silica gel; an activated alumina; a bentonite clay; calcium sulfate; magnesium sulfate; or sodium chloride. In some aspects, the one or more humidity stabilizing materials comprises: magnesium chloride; a hydroxylmethyl cellulose composites; a clay composite; a silica gel; or Propadyn.

In some aspects, the adjusting the humidity of the gas sample in step (b) is a result of the gas sample passing through the one or more conditioning materials. In some aspects, the one or more conditioning materials comprises one or more of permanganate salt, silica, permanganate salt on silica, or activated carbon. In some aspects, the one or more conditioning materials comprises permanganate salt on silica. In some aspects, the permanganate salt on silica is a potassium permanganate on silica.

In some aspects, step (a) and step (b) occur substantially simultaneously.

In some aspects, the adjusting the humidity of the gas sample in step (b) decreases the humidity of the gas sample. In some aspects, the adjusting the humidity of the gas sample in step (b) increases the humidity of the gas sample.

In some aspects, the gas sample passes through the tube in step (c).

In some aspects, the adjusting the humidity of the gas sample to conditions equal to or about equal to ambient humidity in step (d) is a result of passing through the tube.

In some aspects, the one or more input analytes comprises a first input analyte, and wherein the one or more readout analytes comprises a first readout analyte, the method further comprising:

f. before step (e), altering the first input analyte chemically, thereby providing the first readout analyte.

In some aspects, step (f) comprises oxidizing the first input analyte. In some aspects, step (f) comprises reducing the first input analyte. In some aspects, step (f) comprises sorbing one or more contaminants. In some aspects, the gas sample has a pH level, and wherein step (f) comprises adjusting the pH level of the gas sample. In some aspects, the gas sample has an ionic charge, and step (f) comprises adjusting the ionic charge of the gas sample. In some aspects, step (f) comprises one or more of oxidizing the first input analyte, reducing the first input analyte, sorbing one or more contaminants, adjusting a pH level of the gas sample, or adjusting an ionic charge of the gas sample.

In some aspects, step (f) follows step (a) and step (b), and step (f) precedes step (c), step (d), and step (e). In some aspects, step (f) follows step (a), and step (f) precedes step (b), step (c), step (d), and step (e). In some aspects, step (c) and step (d) precede step (a) and step (b). In some aspects, step (f) immediately precedes step (b). In some aspects, step (b) immediately precedes step (f). In some aspects, step (b) and step (f) occur substantially simultaneously.

In some aspects, the gas sample is a breath sample from a human or an animal. In some aspects, the gas sample is provided by a pump, a diffusion, or a vacuum. In some aspects, the first input analyte is nitric oxide. In some aspects, the first readout analyte is nitrogen dioxide. In some aspects, the concentration of nitric oxide in the breath sample is determined using the detection or measurement of nitrogen dioxide in step (e).

In some aspects, the one or more input analytes comprises a first input analyte, the one or more readout analyte comprises a first readout analyte, and the first input analyte is the same as the first readout analyte. In some aspects, the gas sample is a breath sample from a human or an animal. In some aspects, the gas sample is provided by a pump, a diffusion, or a vacuum. In some aspects, the first input analyte comprises nitric oxide.

In some aspects, the detecting or measuring one or more readout analytes is performed by a chemoreceptive sensor. In some aspects, the detecting or measuring one or more readout analytes is performed by a metal oxide sensor. In some aspects, the detecting or measuring one or more readout analytes is performed by an electrochemical sensor. In some aspects, the detecting or measuring one or more readout analytes is performed by a chemiresistive sensor.

In one embodiment the technology is a system comprising

an enclosure comprising: one or more of a frit, a filter, or a membrane, and one or more of a permanganate salt, silica, permanganate salt on silica, or activated carbon; and

a tube in fluid communication with the enclosure, wherein the tube comprises one or more of a perfluorosulfonic acid or a polymer or copolymer derived therefrom, a perflurocarboxylic acid or a polymer or copolymer derived therefrom, or a humidity exchange material; and

one or more sensors to detect and/or measure an analyte;
wherein the enclosure is a cartridge or a capsule.

In some aspects, the enclosure defines an inlet. In some aspects, the enclosure defines an outlet.

In some aspects, the one or more of a frit, a filter, or a membrane comprises a first frit, a first filter, or a first membrane, wherein the one or more of a permanganate salt, silica, permanganate salt on silica, or activated carbon comprises a first permanganate salt, a first silica, a first permanganate salt on silica, or a first activated carbon, wherein the one or more of a frit, a filter, or a membrane comprises a second frit, a second filter, or a second membrane, wherein the first permanganate salt, the first silica, the first permanganate salt on silica, or the first activated carbon is disposed between the first frit, the first filter, or the first membrane; and the second frit, the second filter, or the second membrane.

In some aspects, the one or more of a frit, a filter, or a membrane define one or more pores. In some aspects, the one or more of the permanganate salt, silica, permanganate salt on silica, or activated carbon has a particle size, and the one or more pores is less than the particle size of the one or more of the potassium permanganate, silica, potassium permanganate on silica, or activated carbon. In some aspects, the one or more pores have one or more pore sizes are configured to permit a gas sample passage to traverse the one or more of frit, a filter, or a membrane.

In some aspects, the system further comprises a housing, and the housing is configured to provide fluid communication between the enclosure and the tube. In some aspects, the housing is configured to further provide fluid communication between the enclosure and the tube, and the one or more sensors. In some aspects, the system further comprising a pump, a blower, or a fan connected to the housing, wherein the pump, the blower, or the fan is configured advance a gas through the system.

In some aspects, the enclosure is a capsule, wherein the capsule comprises a cap section and a body section, and wherein the cap section and the body section are configured to press fit together. In some aspects, the cap section defines one or more cap holes. In some aspects, the body section defines one or more body holes. In some aspects, the body section defines one or more body holes and the cap section defines one or more cap holes. In some aspects, the one or more cap holes comprises a first cap hole, and the cap section and the body section are press fit together, thereby covering the first cap hole. In some aspects, the one or more body holes comprises a first body hole, and the cap section and the body section are press fit together, thereby covering the first body hole.

In some aspects, the system further comprises one or more of: a pressure sensitive adhesive; a heat sensitive adhesive; a sonic weld; a bond; a two-part adhesive; or a moisture-cure adhesive. In some aspects, the system further comprises one or more humectants. In some aspects, the one or more humectants comprises: polypropylene glycol; glycerin; sodium hexamethyl phosphate; a glycol; a sugar alcohol; or glyceryl triacetate. In some aspects, the system further comprises one or more desiccants. In some aspects, the one or more desiccants comprises: a silica gel; an activated alumina; a bentonite clay; calcium sulfate; magnesium sulfate; or sodium chloride. In some aspects, the system further comprises one or more humidity stabilizing materials. In some aspects, the one or more humidity stabilizing materials comprises: magnesium chloride; a hydroxylmethyl cellulose composites; a clay composite; a silica gel; or Propadyn.

In some aspects, the one or more sensors comprises a chemoreceptive sensor. In some aspects, the one or more sensors comprises a metal oxide sensor. In some aspects, the one or more sensors comprises an electrochemical sensor. In some aspects, the one or more sensors comprises a chemiresistive sensor.

In some aspects, the enclosure comprises the permanganate salt on silica. In some aspects, the permanganate salt on silica is a potassium permanganate.

In one embodiment, the technology is a system comprising an enclosure comprising: one or more of a frit, a filter, or a membrane, and one or more of a permanganate salt, silica, permanganate salt on silica, or activated carbon; and one or more sensors to detect and/or measure an analyte;

wherein the enclosure is a cartridge or a capsule.

In some aspects, the enclosure defines an inlet. In some aspects, the enclosure defines an outlet.

In some aspects, the one or more of a frit, a filter, or a membrane comprises a first frit, a first filter, or a first membrane, the one or more of a permanganate salt, silica, permanganate salt on silica, or activated carbon comprises a first permanganate salt, a first silica, a first permanganate salt on silica, or a first activated carbon, the one or more of a frit, a filter, or a membrane comprises a second frit, a second filter, or a second membrane, and the first permanganate salt, the first silica, the first permanganate salt on silica, or the first activated carbon is disposed between the first frit, the first filter, or the first membrane; and the second frit, the second filter, or the second membrane.

In some aspects, the one or more of a frit, a filter, or a membrane define one or more pores. In some aspects, the one or more of the permanganate salt, silica, permanganate salt on silica, or activated carbon has a particle size, and the one or more pores is less than the particle size of the one or more of the potassium permanganate, silica, potassium permanganate on silica, or activated carbon. In some aspects, the one or more pores have one or more pore sizes are configured to permit a gas sample passage to traverse the one or more of frit, a filter, or a membrane.

In some aspects, the enclosure is a capsule, wherein the capsule comprises a cap section and a body section, and wherein the cap section and the body section are configured to press fit together. In some aspects, the cap section defines one or more cap holes. In some aspects, the body section defines one or more body holes. In some aspects, the body section defines one or more body holes and the cap section defines one or more cap holes. In some aspects, the one or more cap holes comprises a first cap hole, and the cap section and the body section are press fit together, thereby covering the first cap hole. In some aspects, the one or more body holes comprises a first body hole, and the cap section and the body section are press fit together, thereby covering the first body hole.

In some aspects, the system further comprises one or more of: a pressure sensitive adhesive; a heat sensitive adhesive; a sonic weld; a bond; a two-part adhesive; or a moisture-cure adhesive. In some aspects, the system further comprises one or more humectants. In some aspects, the one or more humectants comprises: polypropylene glycol; glycerin; sodium hexamethyl phosphate; a glycol; a sugar alcohol; or glyceryl triacetate. In some aspects, the system further comprises one or more desiccants. In some aspects, the one or more desiccants comprises: a silica gel; an activated alumina; a bentonite clay; calcium sulfate; magnesium sulfate; or sodium chloride. In some aspects, the system further comprises one or more humidity stabilizing materials. In some aspects, the one or more humidity stabilizing materials comprises: magnesium chloride; a hydroxylmethyl cellulose composites; a clay composite; a silica gel; or Propadyn.

In some aspects, the one or more sensors comprises a chemoreceptive sensor. In some aspects, the one or more sensors comprises a metal oxide sensor. In some aspects, the one or more sensors comprises an electrochemical sensor. In some aspects, the one or more sensors comprises a chemiresistive sensor.

In some aspects, the enclosure comprises the permanganate salt on silica. In some aspects, the permanganate salt on silica is a potassium permanganate.

### BRIEF DESCRIPTION OF THE DRAWINGS

In the drawings:

FIG. 1 depicts the performance of a tube comprising one or more of a perfluorosulfonic acid or a polymer or copolymer derived therefrom, a perflurocarboxylic acid or a polymer or copolymer derived therefrom, or a humidity exchange material as a function of flow rate versus percent relative humidity at different tube lengths and diameters.

FIG. 2 shows an illustrative example of a system or method that includes providing a gas sample, adjusting humidity, converting an analyte, adjusting humidity, and measuring the analyte according to an embodiment of the technology.

FIG. 3A shows an illustrative example of a system or method that includes adjusting humidity and converting an analyte using potassium permanganate on a silica gel substrate in a single step, adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the converted analyte.

FIG. 3B shows one embodiment of use of the system of FIG. 3A for determining the concentration of at least one analyte in a gas sample wherein at least a portion of the gas sample is moved through the system with the aid of a pump, blower or fan. In this example, the sample is exhaled breath from an animal. In some embodiments, the sample is exhaled breath from a human.

FIG. 4A shows an illustrative example of a system that includes adjusting humidity using a silica gel, converting an analyte using potassium permanganate on a silica gel substrate, adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the analyte.

FIG. 4B shows an illustrative example of a system that includes adjusting humidity using a silica gel and converting an analyte using potassium permanganate on a silica gel substrate in a single cartridge, adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the analyte.

FIG. 5 shows an illustrative example of a system that includes a first step adjusting humidity and a second step adjusting humidity according to an embodiment of the technology.

FIGS. 6A and 6B show illustrative examples of cartridges, capsules or test strips according to embodiments of the technology.

FIG. 7 depicts the performance of one configuration of the technology compared to two standard configurations that are not capable of sufficiently adjusting humidity.

FIG. 8 depicts an illustrative example of layers in a test strip configured to condition gas in a sample. This is an exploded view.

FIG. 9 depicts another illustrative example of layers in a test strip configured to condition gas in a sample. This is an exploded view.

FIG. 10 depicts an illustrative example of layers in a test strip configured to condition gas in a sample with additional protective layers. This is an exploded view.

FIG. 11 depicts an illustrative example of layers in a test strip configured to condition gas in a sample with additional protective layers, a layer that may be a spacing layer or a flexible layer, and a gas sensing layer. This is an exploded view.

FIG. 12 depicts an illustrative example of layers in a test strip configured to condition gas in a sample with additional protective layers and a gas sensor. This is an exploded view.

FIG. 13 depicts an illustrative example of layers in a test strip configured to condition gas in a sample with multiple layers of conditioning materials wherein n combinations of layers of conditioning materials is possible. This is an exploded view.

FIG. 14 depicts an illustrative example of layers in a test strip configured to condition gas in a sample where the conditioning layers do not overlap the sensing chemistry. This is an exploded view.

FIG. 15 depicts an illustrative example of layers in a test strip configured to condition gas in a sample with a chamber housing the at least one conditioning material(s) and with additional layers and a gas sensor or gas sensing layer. This is an exploded view.

FIG. 16 depicts an illustrative example of layers in a test strip configured to condition a gas in a sample with chamber comprising at least one conditioning material(s) and a gas sensor or gas sensing layer. This is an exploded view.

FIG. 17 depicts an illustrative example of layers in a test strip configured to condition a gas in a sample with a chamber comprising at least one conditioning material(s) and with a cover layer to allow at least one inlet or outlet to enable a gas to enter and exit the conditioning chamber, and a gas sensor. This is an exploded view.

FIG. 18 depicts an illustrative example of layers in a test strip configured to condition gas in a sample with additional protective layers and a gas sensor or gas sensing layer, and where the gas is passed through the conditioning material and the test strip, and redirected through a tube comprising one or more of a perfluorosulfonic acid or a polymer or copolymer derived therefrom, a perflurocarboxylic acid or a polymer or copolymer derived therefrom, or a humidity exchange material, passed through layers of the test strip to the sensing chemistry. This is an exploded view.

FIG. 19 depicts an illustrative example of layers in a test strip configured to condition gas in a sample with additional protective layers and a gas sensor or gas sensing layer that is housed in a device, and where the gas is passed into the chamber in the device, into the test strip, through the conditioning material in the test strip and the remaining layers of the test strip, out of the chamber in the device, through a tube comprising one or more of a perfluorosulfonic acid or a polymer or copolymer derived therefrom, a perflurocarboxylic acid or a polymer or copolymer derived therefrom, or a humidity exchange material, back into to the chamber in the device, through the layers of the test strip to the sensing chemistry, wherein the inlet and outlet of the tube comprising one or more of a perfluorosulfonic acid or a polymer or copolymer derived therefrom, a perflurocarboxylic acid or a polymer or copolymer derived therefrom, or a humidity exchange material is in fluid communication to the chamber in the device and wherein the tube comprising one or more of a perfluorosulfonic acid or a polymer or copolymer derived therefrom, a perflurocarboxylic acid or a polymer or copolymer derived therefrom, or a humidity exchange material is outside of the chamber in the device. This is an exploded view.

FIG. 20 depicts an illustrative example of layers in a test strip configured to condition gas in a sample with additional protective layers and a gas sensor or gas sensing layer, and where the conditioned gas is directed down a channel formed by the flexible layers and over a sensor. This is an exploded view.

FIG. 21 depicts an illustrative example of various inlet and outlet configurations of a gas conditioning cartridge, capsule, test strip, or test strip chamber.

FIG. 22 depicts an illustrative example of a gas conditioning cartridge or capsule.

FIG. 23 depicts one embodiment of a gas conditioning cartridge or capsule.

FIG. 24 depicts one embodiment of an integrated gas conditioning test strip comprising a chamber, multiple flexible layers, conditioning materials, and optionally a sensor or sensing layer. This is an exploded view.

FIG. 25 depicts an illustrative example of layers in a test strip configured to condition gas in a sample with additional protective layers and a gas sensor that is housed in a device, and where the gas is passed into the chamber in the device, into the test strip, through the conditioning material where it is chemically altered, through the remaining layers of the test strip, out of the chamber in the device, through a tube comprising one or more of a perfluorosulfonic acid or a polymer or copolymer derived therefrom, a perflurocarboxylic acid or a polymer or copolymer derived therefrom, or a humidity exchange material, back into to the chamber in the device, through the layers of the test strip to the sensing chemistry, wherein the inlet and outlet of the tube comprising one or more of a perfluorosulfonic acid or a polymer or copolymer derived therefrom, a perflurocarboxylic acid or a polymer or copolymer derived therefrom, or a humidity exchange material is connected to the chamber in the device and wherein the tube comprising one or more of a perfluorosulfonic acid or a polymer or copolymer derived therefrom, a perflurocarboxylic acid or a polymer or copolymer derived therefrom, or a humidity exchange material is outside of the chamber in the device. This is an exploded view.

FIG. 26 depicts an illustrative example similar to previous embodiments but differs in that the gas enters the bottom of the test strip. This is an exploded view.

FIG. 27 depicts an illustrative example of an embodiment similar to those shown in FIGS. 20 and 24 wherein the gas flows through a channel in the sensor to the sensor or sensing chemistry. This is an exploded view.

FIG. 28 depicts an illustrative example an embodiment wherein the combination of membrane, spacing layer, membrane is stacked upon itself n number of times. This is an exploded view

### DETAILED DESCRIPTION

### Definitions

One or more: As used herein, “one or more” means only one a list, any combination of ones of a list, or all of a list.

Cartridge and Capsule: As used herein, a cartridge or capsule is an enclosure comprising at least one hollow cavity that holds at least one of a membrane, filter, frit, material to condition the gas stream. The cartridge or capsule may be any number of shapes and dimension such that it may hold least one of a membrane, filter, frit, conditioning material, or a combination thereof. Examples include but are not limited to squared, rectangular, or cylindrical. The cartridge or capsule may further comprise at least one inlet in fluid communication with the at least one of a membrane, filter, frit, conditioning material. The cartridge or capsule may further comprise at least one outlet in fluid communication with the at least one of a membrane, filter, frit, conditioning material. In some embodiments, the cartridge or capsule is in fluid communication with a device (e.g. a channel, a lumen, a pathway, or a passage). In some embodiments, the cartridge is in fluid communication with a tube made of at one of perfluorosulfonic acids, perfluorocarboxylic acids, and polymers and co-polymers made there of (e.g. Nafion®).

A capsule is made up of two components, a cap or cap section and a body or body section, wherein the cap and body are in fluid communication when fully assembled. In one embodiment, the body or the cap has a slightly larger diameter or dimension than the corresponding body or cap configured so that the body and cap may be snapped or press fit together. In an embodiment, the cap is combined with the body in such a way so as to enclose the at least one of a membrane, filter, frit, and conditioning material. The cap or body of the capsule may also define holes to enable air to escape when the cap and body are press fit together during manufacturing. The cap holes or body holes may be cover, sealed, and/or occluded when the body and cap are press fit together.

Inside each cartridge or capsule, there may contain a combination of filters, membranes, or frits to encapsulate a liquid, powder or gel material. The material selected to condition the gas stream such that the material chemically reacts, dehumidifies, humidifies or otherwise changes the gas stream as described in examples throughout this document. The cartridge or capsule further may define ridges or internal structures to provide support for the filter, membrane or frit. The walls of the capsule body or cap may further define at least one hole to enable the sample to traverse the capsule. Additional holes may be added to aid in the manufacturing process so that air may escape when the cap and body are joined via a high-speed manufacturing process.

Examples of materials suitable to condition the gas stream in the system include but is not limited to:
Desiccants, including but not limited to silica gels, activated alumina, bentonite clay, calcium sulfate, magnesium sulfate, sodium chloride, or combinations of these;
Sorbents, including but not limited to aluminum oxides, cellulose, polypropylene, molecular sieve, activated carbon, zeolites, carbon nanotubes, clay, bentonite clay, ceramic oxides, silica gel, or combinations of these;
Humectants including but not limited to polypropylene glycol, glycerin, sodium hexamethyl phosphate, glycols, sugar alcohols, glyceryl triacetate, or combinations of these;
Dynamic humidity equilibrators including but not limited to magnesium chloride, hydroxylmethyl cellulose composites, clay composites, silica gel, Propadyn®, or combinations of these;
Humidity exchange materials including but not limited to perfluorosulfonic acid, perflurocarboxylic acid, polymers and co-polymers of perfluorosulfonic acid, polymers and co-polymers of perfluorosulfonic acid, or combinations of these;
Chemically modifying materials including but not limited to permanganate salts, potassium permanganate, sodium permanganate, permanganate salt on silica gel, permanganate salt on alumina, permanganate salt supported on a solid or porous particulate, permanganate salt supported on a porous mesh or filter, silica gel, silica nanoparticles, gold nanoparticles, nanoparticles, palladium powders, platinum powders, catalytic metals and metal oxides, reducing agents, oxidizing agents, complexing agents, ion exchange resins, pH modifiers, other chemically active species known in the art for converting or changing chemical species or combinations thereof, or combinations of these.

Membranes, filters or frits may also serve as suitable materials to condition the gas stream and/or to encapsulate materials suitable to condition the gas stream. The membrane may condition the gas stream in a number of ways including but not limited to: selectively allowing certain species to pass through, allowing only species below a size threshold through, filtering particulate, preventing species above a certain size threshold from passing through, oxidizing, reducing, humidifying, dehumidifying, equilibrating with ambient conditions, heating, cooling, chemically complexing, condensing to a liquid, condensing to a solid, adjusting the pH, converting from a liquid to a gas, converting from a solid to a liquid or gas, change the chemical state, change the physical state, or any combination thereof. Examples of suitable membrane or filter materials include but are not limited to polypropylene, nylon, polyester, polyethylene, PTFE, PET, natural fibers, cellulose, fiberglass, activated charcoal, cotton, polyethersulfone, polyurethanes, foams, polycarbonate, polystyrene, perfluorosulfonic acid polymers and co-polymers, perfluorocarboxylic acid polymers and co-polymers, Nafion®, or other membrane, or filtration materials know in the art to be chemically compatible with, and of small enough pore size to prevent migration of selected conditioning materials. The membrane or filter herein can be of any size or thickness required for a particular sensing application. For example, in some embodiments a membrane, or filter for a test strip may have dimensions less than 200 cm² and a thickness less than 1 cm. In other embodiments, the membrane or filter on a test strip may be less than 1 cm wide by 10 cm long, with a thickness of less than 5 mm. In other embodiments, the membrane, or filter in a cartridge spans the entire length and width of the interior of the cartridge, with a thickness of less than 5 mm. In other embodiments, the membrane, or filter on a test strip. In some embodiments, the membrane, filter or frit is sufficiently porous to capture the material to condition the gas stream while enabling gas to pass through it.

Examples of suitable frit materials include but are not limited to UHMW, Polyethylene or PE copolymers, glass, quartz, polytetrafluoroethylene (PTFE), aluminum oxide, ceramics, and other materials known in the art. Examples include frits used in chromatography such as those supplied by GenPore—A Division of General Polymeric Corporation. In some embodiments, frits have a pore size between 5-50 microns. In some embodiments, the frits may be configured in hydrophobic or hydrophilic formulations. In some embodiments, the frits are wide enough to span the width of a tube, cartridge, capsule, test strip, or test strip chamber. In some embodiments, the frits are press fit into the cartridge, capsule, test strip, or test strip chamber. In some embodiments, the frits are less than 5 cm in diameter. In some embodiments, the frits are less than 1 cm in diameter. In some embodiments, the frits are thick enough to prevent migration of powdered conditioning materials. In some embodiments, the frits have a pore size between 1-5 microns.

Test Strip: A test strip is well known in the art for use in medical diagnostics, life sciences, or environmental sciences. Examples include but are not limited to glucose sensors, lateral flow strips and cartridges, as well as for test strips detecting creatinine, ketone, lactate, INR etc. This is not intended to be an exhaustive list. Test strips may also include gas sensors as previously described by the authors. In this context a test strip may contain a combination of flexible layers, and further contain elements for condition a gas. Materials may be chosen to ensure low cost, flexibility, ease of use, or chemical compatibility with the conditioning materials, analytes of interest, or any associated sensors or sensor test strips. Test strips may be comprising various combinations of flexible layers. Examples of test strip materials include, but are not limited to polyester, polyimide (e.g. under the brand name Kapton®), PET, polypropylene, polyethylene, thermoplastics, silicone, silicone or acrylic adhesives, medical tapes, and other materials known in the art of test strips and cartridges for use in medical diagnostics, life or environmental sciences. Examples of suitable materials are those provided by Tekra (e.g. under the brand name Melinex®), 3M, Adhesives Research or TekPak. This is not intended to be an exhaustive list. Any combination of the layers of the test strip may be bound together by additional layers such as pressure or heat sensitive adhesives. Examples include, but are not limited to, silicone and acrylic adhesives. Layers may also be bound together by other techniques such as, thermal bonding, sonic welding, two-part adhesives, moisture cure adhesives, and other techniques know to those in the art.

Layers of the test strip may be processed to create features such as partial or thru holes, channels, indentations, single or multiple holes. The holes may be filled with material to condition the gas stream. Holes may also be tapered. In some embodiments, the tapered hole is gradually smaller or narrowed at one end. In some embodiments, the tapered hole has a first diameter on a first surface of the layer, and the tapered hole has a second diameter on a second surface of the layer, where the second diameter is less than the first diameter. In another embodiment, the second diameter is greater than the first diameter. Various degrees or angles of taper are possible without deviating from the spirit of the technology. Tapering the hole enables more efficient filling of the hole with a material to condition the gas stream during manufacturing. Tapered holes are possible in any of the described configurations.

The test strip may also contain a sensing layer comprising of at least one electrode disposed on a substrate. In this embodiment, the substrate is made of at least one flexible layer. The sensing layer may also contain at least one sensing chemistry. In some embodiments, the sensing chemistry is configured to bridge the at least one electrode. The sensor or sensing chemistry may be configured to sense any number of analytes in the gas stream or the product of any chemical or physical modifications that have been made by the gas conditioning system.

Foil or other gas impermeable barriers may be incorporated into the test strip, test strip chamber, test strip layers, capsule or device. In some embodiments, the device punctures this foil layer or barrier.

As used herein, a “gas sample receiver” may refer to a cartridge, a capsule, a test strip or a test strip chamber. In some embodiments, the gas sample receiver is at least one of single use, limited use, disposable, reusable, able to be regenerated, or unlimited use.

Sensors: Many types of sensors for analyte detection are known in the art and may be used in the system described herein. Examples include but are not limited to: metal oxide sensors (MOS, CMOS, etc.), electrochemical sensors, MEMS sensors, acoustic sensors, Infra-Red sensors, laser sensors, colorimetric, chemiluminescence, GC/MS, Field Asymmetric Ion Mobility sensor, graphene sensors, optical, FET, MOSFET, and ChemFET sensors, chemoreceptive sensor, chemiresistive sensors, and sensors previously described in International Patent Application Numbers PCT/US2015/000180, PCT/US2015/034869, and PCT/US2017/042830, incorporated by reference in their entireties. Any appropriate sensing layer or sensing chemistry in may be replaced by a sensor known in the art.

Sensing Chemistry: Many sensing chemistries are possible without deviating from the spirit of the technology. In one embodiment, the sensing chemistry is comprising nanostructures functionalized to bind to an analyte causing an electrical resistance change across the nanostructures. In other embodiments the analyte causes a redox reaction at the nanostructural level, which is measured. In another embodiment, the analyte causes a change in the surface electrons of the sensing chemistry, resulting in changes in the optical characteristics, which are measured. Nanostructures may include, but are not limited to, carbon nanotubes (single walled, multiwalled, or few-walled), nanowires, graphene, graphene oxides, etc. The nanostructures can be assembled to form macroscopic features, such as papers, foams, films, etc. or may be embedded in or deposited on macrostructures. Examples of functionalization materials include, but are not limited to:
Heterocyclic macrocycles including, but are not limited to crown ethers, phthalocyanines, porphyrins, etc., or a combination thereof;
Metal oxides including, but are not limited to AgO, CeO₂, Co₂O₃, CrO₂, PdO, RuO₂, TiO₂, or a combination thereof;
Transition metals including, but are not limited to, Ag, Cu, Co, Cr, Fe, Ni, Pt, Ru, Rh, Ti, or a combination thereof;
Carboxyl groups including, but are not limited to carboxylic acids;
Functional Organic Dyes including, but are not limited to, Azo dyes, Cyanines, Fluorones, indigo dyes, photochromic dyes, Phthalocyanines Xanthens, etc., or a combination thereof;
or combinations of these.

The functionalized nanostructure, hereafter referred to as sensing chemistry, is disposed over a substrate or flexible substrate to form the basic components of a sensing layer. Electrodes may be in electrical communication with the sensing chemistry.

In another embodiment, the sensing chemistry is a non-functionalized (i.e. un-sensitized) nanostructure. This embodiment may be used in conjunction with a functionalized nanostructure or it may stand-alone.

Secondary additives may be used to affect the drying characteristics and process ability of the sensing chemistry for deposition onto a substrate. Non limiting examples of deposition methods include: Air knife coating, Inkjet, Curtain coating, Knife over roll (tape casting), Dip coating, Lamination, Doctor blade, Meyers rod coating, Drop casting, Offset Electropainting, Pad printing, Electrophoretic deposition, Press Fitting, Electrospray, Roll coating, Flexography, Rotary screen, Gravure, Screen, Hot melt, Slot-die, Ink rolling, Spin coating, Spray coating, or any other method known in the art. Additives may be used to change the viscosity, surface tension, wettability, adhesion, drying time, gelation, film uniformity, etc. These additives include, but are not limited to, secondary solvents, thickeners, polymers, salts, and/or surfactants. These additives may serve one or multiple purposes. Examples may include, but are not limited to:
Thickeners—polymeric and non-polymeric—including, but not limited to, Glycerol
Polypropylene glycol, or any combination thereof;
Surfactants—ionic and non-ionic—including, but not limited to Sodium dodecyl sulfate, Triton X-100, or any combination thereof;
Additives including, but not limited to Alkyltrimethylamminumsalts,
Anionicsurfactants, Cationicsurfactants, Cellulosics, Clays, Ethyleneglycol, Fluorosurfactants, Glycerol, Nonionicsurfactants, Organicsolvents, Polyacrylicacid, Polyoxyethylenenonylphenylether, Polysaccharides, Polyurethanes, Polyvinyl butyral, Proteins, Silica, Silicones, Sodiumdodecylsulfate, Stearicacid, Water, Zwitterionicsurfactants, or any combination thereof;
Or any combinations of these.

In some embodiments, the volume of sensing chemistry disposed on the substrate maybe less than or equal to 1 milliliter of material.

Device or Housing: As used herein, a device (e.g. a channel, a lumen, a pathway, or a passage) comprises a gas sample inlet and a chamber within the device configured to house at least one of a test strip, test strip chamber, cartridge, or capsule. The device chamber may contain any number of inlets and outlets to match the appropriate configuration of the cartridge, capsule, test strip, test strip chamber, or sensor. In some embodiments, the device chamber is not fully enclosed. In some embodiments, the device chamber defines a slot-opening. In some embodiments, the device chamber is open on one surface. In some embodiments, the chamber within the device is configured to enable easy removal of the cartridge, capsule, test strip, or test strip chamber. In one embodiment, the device further contains a tube comprising at least one of perfluorosulfonic acids, perfluorocarboxylic acids, and polymers and co-polymers made there of (e.g. Nafion®). In one embodiment, the chamber within the device is further configured to enable fluid communication between the gas sample inlet, at least one of a test strip, test strip chamber, cartridge, or capsule, tube, and at least one of a sensor or sensing chemistry. The device may contain a gas sample outlet. The device may be comprising a combination of a display screen, pump, power supply, wireless radio (e.g. non exhaustive list: Bluetooth, Wi-Fi, NFC, or cellular), uv source, plasma source, sensors to measure pressure, flow rate, temperature, humidity, accelerometer, or LED. The device may also be configured to alter the temperature, humidity, chemical make up, pressure of the gas stream. Alterations to the gas may be any combination of increase, decrease, equilibrate at least one of temperature, pressure, and humidity. This is not intended to be an exhaustive list.

Selective Membrane: As used herein, a selective membrane means a membrane that allows specific species to pass through it (e.g. a sodium selective membrane is configured to only or chiefly only allow sodium ions to traverse). A humidity exchange material is a selective membrane that allows moisture in the gas stream to pass in either direction, resulting in an equilibration of humidity between the gas sample and the ambient environment. A size exclusion membrane is a selective membrane that allows only or chiefly only particles or molecules below a preselected size threshold to pass through, preventing larger species to pass through. Size exclusion membrane may be used primarily as a membrane configured to allow species smaller than about 1 micron to pass through. A particulate filter is a selective membrane similar to a size exclusion membrane. Particular filter membranes may be used when dealing with larger particles (e.g. greater than 1 micron).

Embodiments of this technology include methods and systems for conditioning gas for analysis and determining the concentration of at least one analyte in a gas sample. In general, determining the concentration of an analyte in a gas sample includes a combination and/or repetition of steps related to dehumidifying and/or humidifying the gas, and/or performing a chemical reaction on at least one analyte and measuring the product of the chemical reaction or measuring the at least one analyte without performing a chemical reaction. In one embodiment of the technology, a chemical reaction is used to remove an interferent from the gas sample. In another embodiment, the system is configured to dehumidify, chemically alter and equilibrate the sample to ambient humidity. Other aspects of the technology may also alter the temperature of the gas. In one embodiment of the technology, the method is related to measuring an analyte or analytes in exhaled breath. In one embodiment, the system is configured to measure nitric oxide in exhaled breath. In another embodiment of the system is configured to oxidize nitric oxide into nitrogen dioxide in exhaled breath. Other non-breath examples include analytes for the environmental, fire and safety, defense/military, automotive, industrial, and agricultural industries.

One aspect of the technology involves a low-cost sensor and methods to condition an analyte in a breath sample.

In another aspect of the technology, a system for conditioning at least one analyte in a gas sample is disclosed, in which the system comprises a cartridge, capsule, test strip, or, test strip chamber for adjusting humidity and a tube comprising one or more of a perfluorosulfonic acid or a polymer or copolymer derived therefrom, a perflurocarboxylic acid or a polymer or copolymer derived therefrom, or a humidity exchange material.

In another aspect of the technology, a system for determining the concentration of at least one analyte in a gas sample is disclosed, in which the system comprises a cartridge, capsule, test strip, or test strip chamber for adjusting humidity, a tube comprising one or more of a perfluorosulfonic acid or a polymer or copolymer derived therefrom, a perflurocarboxylic acid or a polymer or copolymer derived therefrom, or a humidity exchange material, and a sensor. In some embodiments, the cartridge, capsule, test strip, or test strip chamber is configured to accept a gas sample from a human user as previously described in the applications incorporated above.

In another aspect of the technology, a method for conditioning at least one analyte in a gas sample is disclosed, in which the method comprises adjusting humidity and converting at least one analyte. In some embodiments, adjusting humidity and converting at least one analyte occurs in a single step.

In one embodiment, a method for determining the concentration of at least one analyte in a gas sample is disclosed, in which the method comprises adjusting the humidity, converting the analyte, adjusting the humidity, and measuring the analyte.

In one embodiment, a method for determining the concentration of at least one analyte in a gas sample is disclosed, in which the method comprises converting the analyte and adjusting humidity in a single step, adjusting humidity in a second step, and measuring the analyte. In some embodiments, adjusting humidity comprises at least one of dehumidifying, humidifying, and equilibrating to ambient relative humidity.

In one embodiment, a method for determining the concentration of at least one analyte in a gas sample is disclosed, in which the method comprises converting the analyte and adjusting humidity in a single step using at least one of a permanganate salt on silica gel, adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the analyte using a sensor. In some embodiments, adjusting humidity comprises at least one of dehumidifying, humidifying, and equilibrating to ambient relative humidity. In some embodiments, the method comprises converting the analyte and adjusting humidity in a single step using at least one of potassium and sodium permanganate on silica gel, adjusting humidity using a tube comprising one or more of a perfluorosulfonic acid or a polymer or copolymer derived therefrom, a perflurocarboxylic acid or a polymer or copolymer derived therefrom, or a humidity exchange material wherein the sample returns to ambient conditions, and measuring the analyte using a sensor.

In one embodiment, a method for determining the concentration of at least one analyte in a gas sample is disclosed, in which the method comprises adjusting humidity using a tube comprising one or more of a perfluorosulfonic acid or a polymer or copolymer derived therefrom, a perflurocarboxylic acid or a polymer or copolymer derived therefrom, or a humidity exchange material, converting the analyte and adjusting humidity in a single step using at least one of potassium and sodium permanganate on silica gel, and measuring the analyte using a sensor. In some embodiments, adjusting humidity comprises at least one of dehumidifying, humidifying, and equilibrating to ambient relative humidity. In some embodiments, the method comprises adjusting humidity using a tube comprising one or more of a perfluorosulfonic acid or a polymer or copolymer derived therefrom, a perflurocarboxylic acid or a polymer or copolymer derived therefrom, or a humidity exchange material (e.g. a Nafion® tube), converting the analyte and adjusting humidity in a single step using at least one of potassium and sodium permanganate on silica gel wherein the sample returns to ambient conditions, and measuring the analyte using a sensor.

In one embodiment, a method for determining the concentration of at least one analyte in a gas sample is disclosed, in which the method comprises adjusting humidity using a tube comprising one or more of a perfluorosulfonic acid or a polymer or copolymer derived therefrom, a perflurocarboxylic acid or a polymer or copolymer derived therefrom, or a humidity exchange material, converting the analyte and adjusting humidity in a single step using at least one of potassium and sodium permanganate on silica gel, and measuring the analyte using a sensor. In some embodiments, adjusting humidity comprises at least one of dehumidifying, humidifying, and equilibrating to ambient relative humidity. In some embodiments, the method comprises adjusting humidity using a tube comprising one or more of a perfluorosulfonic acid or a polymer or copolymer derived therefrom, a perflurocarboxylic acid or a polymer or copolymer derived therefrom, or a humidity exchange material, converting the analyte and adjusting humidity in a single step using at least one of potassium and sodium permanganate on silica gel wherein the sample returns to ambient conditions, and measuring the analyte using a sensor.

In one embodiment, a method for determining the concentration of at least one analyte in a gas sample is disclosed, in which the method comprises adjusting humidity using a silica gel, converting the analyte using at least one of potassium and sodium permanganate on silica gel, and measuring the analyte using a sensor. In some embodiments, adjusting humidity comprises at least one of dehumidifying, humidifying, and equilibrating to ambient relative humidity. In some embodiments, the method comprises adjusting humidity using a silica gel wherein the sample returns to ambient conditions, converting the analyte using at least one of potassium and sodium permanganate on silica gel, and measuring the analyte using a sensor.

In one embodiment, a method for determining the concentration of at least one analyte in a gas sample is disclosed, in which the method comprises adjusting humidity using a silica gel, converting the analyte using at least one of potassium, and sodium permanganate, and a silica gel functionalized with at least one of potassium and sodium permanganate, and measuring the analyte using a sensor. In some embodiments, adjusting humidity comprises at least one of dehumidifying, humidifying, and equilibrating to ambient relative humidity. In some embodiments, the method comprises adjusting humidity using a silica gel wherein the sample returns to ambient conditions, converting the analyte using at least one of potassium, and sodium permanganate, and a silica gel functionalized with at least one of potassium and sodium permanganate.

In one embodiment, a method for determining the concentration of at least one analyte in a gas sample is disclosed, in which the method comprises adjusting humidity using a silica gel, converting the analyte using at least one of potassium and sodium permanganate optionally on a silica gel substrate, adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the analyte with a sensor. In some embodiments, adjusting humidity comprises at least one of dehumidifying, humidifying, and equilibrating to ambient relative humidity. In some embodiments, the method comprises adjusting humidity using a silica gel, converting the analyte using at least one of potassium and sodium permanganate optionally on a silica gel substrate, adjusting humidity using a tube comprising one or more of a perfluorosulfonic acid or a polymer or copolymer derived therefrom, a perflurocarboxylic acid or a polymer or copolymer derived therefrom, or a humidity exchange material wherein the sample returns to ambient conditions, and measuring the analyte with a sensor.

In one embodiment, a method for determining the concentration of at least one analyte in a gas sample is disclosed, in which the method comprises a first step adjusting humidity, a second step adjusting humidity, and measuring the analyte. In some embodiments, the first step adjusting humidity comprises at least one of dehumidifying, humidifying, and equilibrating to ambient relative humidity, such as through the use of desiccants (e.g. silica gel, clay desiccants), humectants (e.g. propylene glycol, glycerin, sodium hexametaphosphate, etc.), dynamic chemical stabilizers (e.g. Propadyn® as disclosed in European Patent Number 2,956,237B1, incorporated by reference in its entirety, a MgCl2/hydroxypropylmethyl cellulose composite material), or a tube comprising one or more of a perfluorosulfonic acid or a polymer or copolymer derived therefrom, a perflurocarboxylic acid or a polymer or copolymer derived therefrom, or a humidity exchange material. In some embodiments, the second step adjusting humidity comprises at least one of dehumidifying, humidifying, and equilibrating to ambient relative humidity.

In one embodiment, a method for determining the concentration of at least one analyte in a gas sample is disclosed, in which the method comprises a first step adjusting humidity using a silica gel, a second step adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the analyte. In some embodiments, the first step adjusting humidity comprises at least one of dehumidifying, humidifying, and equilibrating to ambient relative humidity. In some embodiments, the second step adjusting humidity comprises at least one of dehumidifying, humidifying, and equilibrating to ambient relative humidity.

In one embodiment, a method for determining the concentration of at least one analyte in a gas sample is disclosed, in which the method comprises a first step adjusting humidity using a tube comprising one or more of a perfluorosulfonic acid or a polymer or copolymer derived therefrom, a perflurocarboxylic acid or a polymer or copolymer derived therefrom, or a humidity exchange material, a second step adjusting humidity using a silica gel, and measuring the analyte. In some embodiments, the first step adjusting humidity comprises at least one of dehumidifying, humidifying, and equilibrating to ambient relative humidity. In some embodiments, the second step adjusting humidity comprises at least one of dehumidifying, humidifying, and equilibrating to ambient relative humidity.

In another aspect of the technology, a method for determining the concentration of at least one analyte in a gas sample is disclosed, in which the method comprises adjusting humidity, converting at least one analyte, and measuring the at least one analyte. In some embodiments, adjusting humidity and converting at least one analyte occurs in a single step.

In some embodiments, a silica gel adjusts humidity. In some embodiments, a functionalized silica gel adjusts humidity. In some embodiments, a tube comprising one or more of a perfluorosulfonic acid or a polymer or copolymer derived therefrom, a perflurocarboxylic acid or a polymer or copolymer derived therefrom, or a humidity exchange material adjusts humidity. In some embodiments, a membrane and a Nafion® tube adjusts humidity. In some embodiments, a chamber or flow path with a large surface area adjusts humidity. In some embodiments, a desiccant, such as sodium chloride, activated alumina, activated charcoal, calcium chloride, bentonite clay, adjusts humidity. In some embodiments, a humectant, such as glycols, alpha hydroxy acids, polyols, and sugar polyols, adjusts humidity. In some embodiments, dynamic chemical stabilizers, such as MgCl2/cellulose composites, Propadyn®, or other humidity equilibration materials, adjusts humidity. In some embodiments, a mechanical or electrical means, such as evaporator and condenser coils, adjusts humidity.

In some embodiments the cartridge, capsule, test strip, or test strip chamber is in fluid communication with the tube. In some embodiments the fluid communication is with at least one of an inlet or outlet defined by the cartridge, capsule, test strip, or test strip chamber. In some embodiments the fluid communication is with at least one of the inlet or outlet of the tube.

In some embodiments, the tube has a length of less than 24 inches. In some embodiments, the tube has a length of less than 18 inches. In some embodiments, the tube has a length of less than 12 inches. In some embodiments, the tube has a length of less than 6 inches.

In some embodiments, the tube has a diameter of less than 0.110 inches. In some embodiments, the tube has a diameter of less than 0.070 inches. In some embodiments, the tube has a diameter of less than 0.060 inches. In some embodiments, the tube has a diameter of less than 0.050 inches. Any of the diameters may be combined with any of the tube lengths described herein.

In some embodiments, the analyte is converted by oxidation. In some embodiments, the analyte is converted by reduction. In some embodiments, the analyte is converted by formation of complexes. In some embodiments, the analyte is converted by covalent bonding. In some embodiments, the analyte is converted by chemical reactions. In some embodiments, the analyte is converted by a change in physical state. In some embodiments, the analyte is condensed into a gas. In some embodiments, the analyte forms a plasma. In some embodiments, the analyte volatilizes a compound. In another aspect of the technology, the analyte is converted by humidity adjustment.

In one embodiment, exhaled nitric oxide is converted into nitrogen dioxide. In one embodiment, hydrogen is converted into at least one of water, reduced organic species, and reduced inorganic species (e.g. reduction of alcohols to hydrocarbons, reduction of metal oxides to metals, etc.). In one embodiment, methane is converted into at least one of hydrocarbon species, ketones, carbonyls, ethers, alcohols, halides, amines, aldehydes, amides, alkaloids, ions, radicals, and other reactive organic species. In one embodiment, ethylene is converted into at least one of hydrocarbon species, ketones, carbonyls, ethers, alcohols, halides, amines, aldehydes, amides, alkaloids, ions, radicals, and other reactive organic species. In some embodiments, exhaled nitric oxide is converted into nitrogen dioxide immediately before, immediately after, or substantially at the same time as the humidity adjustment. In some embodiments, hydrogen is converted into at least one of water, reduced organic species, and reduced inorganic species (e.g. reduction of alcohols to hydrocarbons, reduction of metal oxides to metals, etc.) immediately before, immediately after, or substantially at the same time as the humidity adjustment. In some embodiments, methane is converted into at least one of hydrocarbon species, ketones, carbonyls, ethers, alcohols, halides, amines, aldehydes, amides, alkaloids, ions, radicals, and other reactive organic species immediately before, immediately after, or substantially at the same time as the humidity adjustment. In some embodiments, ethylene is converted into at least one of hydrocarbon species, ketones, carbonyls, ethers, alcohols, halides, amines, aldehydes, amides, alkaloids, ions, radicals, and other reactive organic species immediately before, immediately after, or substantially at the same time as the adjusting humidity. In some embodiments, adjusting humidity comprises at least one of dehumidifying, humidifying, and equilibrating to ambient relative humidity. In some embodiments, adjusting humidity and converting at least one analyte occurs in a single step. In some embodiments, a method for converting exhaled nitric oxide into nitrogen dioxide is disclosed, in which the method comprises a gas sample passing through at least one of potassium permanganate and sodium permanganate suspended on a silica gel.

In some embodiments, potassium permanganate converts the analyte. In some embodiments, sodium permanganate converts the analyte. In some embodiments, functionalized silica gel converts the analyte. In some embodiments, functionalized silica gel comprises at least one of permanganate, potassium permanganate, and sodium permanganate. In other embodiments, a UV source converts the analyte. In other embodiments, an infrared source converts the analyte. In other embodiments, a radio frequency source converts the analyte. In other embodiments, a corona discharge source converts the analyte.

In some embodiments, the analyte is measured by a sensing technology known in the art. In some embodiments, the analyte is measured by sensors as previously described in the applications incorporated above. In some embodiments, the analyte is measured by metal oxide sensors (MOS, CMOS, etc.). In some embodiments, the analyte is measured by electrochemical sensors. In some embodiments, the analyte is measured by MEMS sensors. In some embodiments, the analyte is measured by acoustic sensors. In some embodiments, the analyte is measured by IR sensors. In some embodiments, the analyte is measured by laser sensors. In some embodiments, the analyte is measured by chemiluminescence. In some embodiments, the analyte is measured by GC/MS sensors. In some embodiments, the analyte is measured by Field Asymmetric Ion Mobility sensors. In some embodiments, the analyte is measured by graphene sensors. In some embodiments, the analyte is measured by electrochemical sensors. In some embodiments, the analyte is measured by optical sensors. In some embodiments, the analyte is measured by FET, MOSFET, and ChemFET sensors. In some embodiments, the analyte is measured by chemiresistive sensors.

In some embodiments, the gas sample is at least one of heated or cooled. In some embodiments, the difference between the relative humidity of the sample and ambient conditions is about 3% RH. In some embodiments, the difference between the relative humidity of the sample and ambient conditions is less than 5% RH. In some embodiments, the difference between the relative humidity of the sample and ambient conditions is less than 10% RH. In some embodiments, the difference between the relative humidity of the sample and ambient conditions is less than 15% RH. In some embodiments, the difference between the relative humidity of the sample and ambient conditions is less than 20% RH.

In some embodiments, the analyte is converted in the form of a cartridge, capsule, test strip, or test strip chamber and measured by a sensor as previously described in International Patent Application Numbers PCT/US2015/000180, PCT/US2015/034869, and PCT/US2017/042830, hereby incorporated by reference in their entirety. In one embodiment, the cartridge, capsule, test strip, or test strip chamber uses a powdered substance for adjusting humidity. In one embodiment, the cartridge, capsule, test strip, or test strip chamber uses a powdered substance for converting the analyte. In one embodiment, the cartridge, capsule, test strip, or test strip chamber contains at least one of a permeable and semi-permeable material to hold the conversion media in place. In one embodiment, the cartridge, capsule, test strip, or test strip chamber contains at least one of a permeable and semi-permeable material to enable the flow of gas through the cartridge, capsule, test strip, or test strip chamber and is powdered media. In some embodiments, the cartridge, capsule, test strip, or test strip chamber comprises at least one of polymers, composite materials, fibrous materials such as paper or fiber glass, woven and non-woven textiles, membranes, ceramics, metals, metal oxides, glasses, sintered materials, etched materials, perforated materials, and other gas porous or permeable materials. In some embodiments, the cartridge, capsule, test strip, or test strip chamber comprises frits. In some embodiments, the at least one of the permeable and semi-permeable material also aids in adjusting humidity. In some embodiments, the at least one of the permeable and semi-permeable material also aids in adjusting the flow rate. In some embodiments, the outer structure of the cartridge, capsule, test strip, or test strip chamber enables a connection to the flow path of the gas. In some embodiments, the cartridge, capsule, test strip, or test strip chamber is reusable. In some embodiments, the cartridge, capsule, test strip, or test strip chamber is semi-reusable. In some embodiments, the cartridge, capsule, test strip, or test strip chamber is single use. In some embodiments, the cartridge, capsule, test strip, or test strip chamber is disposable. In some embodiments, the cartridge, capsule, test strip, or test strip chamber is removable from the system. In some embodiments, the cartridge, capsule, test strip, or test strip chamber is not removable from the system.

In some embodiments, the cartridge, capsule, test strip, or test strip chamber contains less than or equal to 5 g of potassium permanganate or sodium permanganate. In some embodiments, the cartridge, capsule, test strip, or test strip chamber contains less than or equal to 1 g of potassium permanganate or sodium permanganate. In some embodiments, the cartridge, capsule, test strip, or test strip chamber contains less than or equal to 0.5 g of potassium permanganate or sodium permanganate. In some embodiments, the cartridge, capsule, test strip, or test strip chamber contains less than or equal to 0.1 g of potassium permanganate or sodium permanganate. In some embodiments, the cartridge, capsule, test strip, or test strip chamber contains less than or equal to 0.01 g of potassium permanganate or sodium permanganate.

In some embodiments, the cartridge, capsule, test strip, or test strip chamber contains less than or equal to 5 g of potassium permanganate or sodium permanganate on a silica gel (e.g., functionalized silica). In some embodiments, the cartridge, capsule, test strip, or test strip chamber contains less than or equal to 1 g of potassium permanganate or sodium permanganate. In some embodiments, the cartridge, capsule, test strip, or test strip chamber contains less than or equal to 0.5 g of potassium permanganate or sodium permanganate on a silica gel (e.g., functionalized silica). In some embodiments, the cartridge, capsule, test strip, or test strip chamber contains less than or equal to 0.1 g of potassium permanganate or sodium permanganate on a silica gel (e.g., functionalized silica). In some embodiments, the cartridge, capsule, test strip, or test strip chamber contains less than or equal to 0.01 g of potassium permanganate or sodium permanganate on a silica gel (e.g., functionalized silica).

In some embodiments, the cartridge, capsule, test strip, or test strip chamber dimensions of any one of length, width, or height is less than or equal to 7.62 cm. In some embodiments, cartridge, capsule, test strip, or test strip chamber is cylindrical wherein the dimensions of any one of length or diameter is less than or equal to 7.62 cm. In some embodiments, the cartridge, capsule, test strip, or test strip chamber is cylindrical wherein the dimensions of any one of length or diameter is less than or equal to 2.54 cm. In another embodiment, the cartridge, capsule, test strip, or test strip chamber is cylindrical with a length of less than or equal to 2.54 cm and a radius of less than, or equal to 1.27 cm. In another embodiment, the cartridge, capsule, test strip, or test strip chamber is cylindrical with a length of less than or equal to 1.5 cm and a radius of less than or equal to 1 cm. In another embodiment, the cartridge, capsule, test strip, or test strip chamber is cylindrical with a length of less than or equal to 1.5 cm and a radius of less than or equal to 0.5 cm. In another embodiment, the cartridge, capsule, test strip, or test strip chamber is cylindrical with a length of less than or equal to 1.5 cm and a radius of less than or equal to 2 cm. In another embodiment, the cartridge, capsule, test strip, or test strip chamber is cylindrical with a length of less than or equal to 1 cm and a radius of less than or equal to 2 cm.

In some embodiments, the gas sample moves through the system with the aid of at least one of a pump, a blower or a fan. In some embodiments, the pump samples a side stream from a main gas stream as previously described in the applications incorporated above. In some embodiments, the blower samples a side stream from a main gas stream as previously described in the applications incorporated above.

### Examples

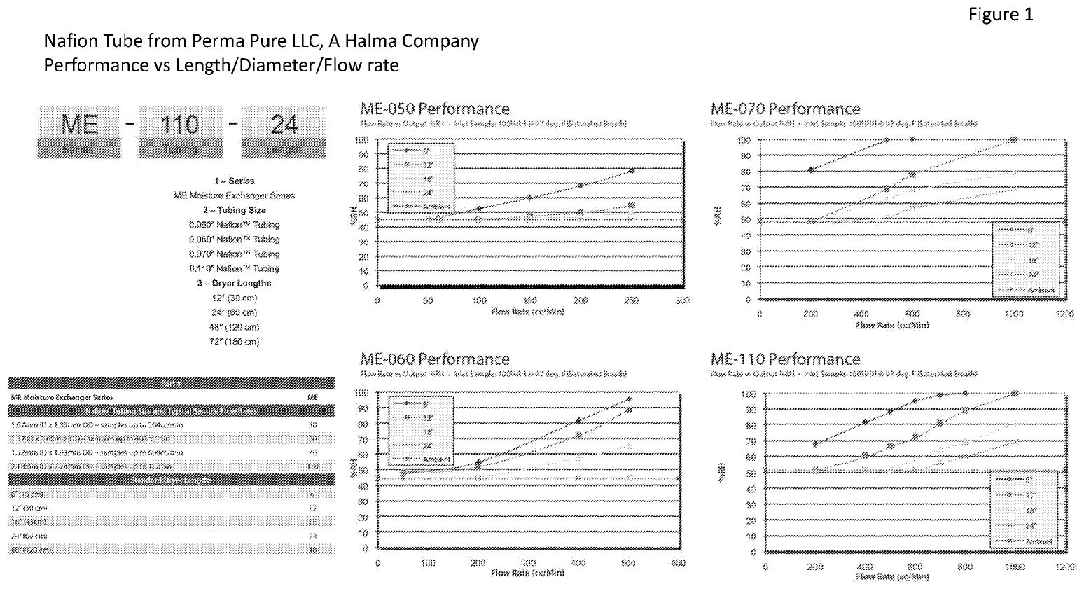

FIG. 1 depicts the performance of Nafion® tube at different flow rates where the sample inlet is saturated breath. The efficiency of the Nafion® tube to humidify or dehumidify is dependent upon its length, inner diameter, outer diameter, and the flow rate of the gas. The higher the flow rate, the longer the length and larger diameter the Nafion® tube must be to equilibrate the sample with ambient conditions. For example, Nafion® tubes from Perma Pure LLC, A Halma Company ME Moisture Exchanger Series with inner diameters of 1.07 mm, 1.32 mm, 1.52 mm, and 2.18 mm, and outer diameters of 1.35 mm, 1.60 mm, 1.83 mm, and 2.74 mm respectively will differ in percent of relative humidity removed from a breath sample at higher flow rates. Similarly, Nafion® tubes with lengths of 6 inches, 12 inches, 18 inches, and 24 inches will differ in percent relative humidity removed from a breath sample at higher flow rates. Nafion® tubes of smaller diameters and smaller lengths perform better at lower flow rates while larger diameters and longer lengths perform better at higher flow rates.

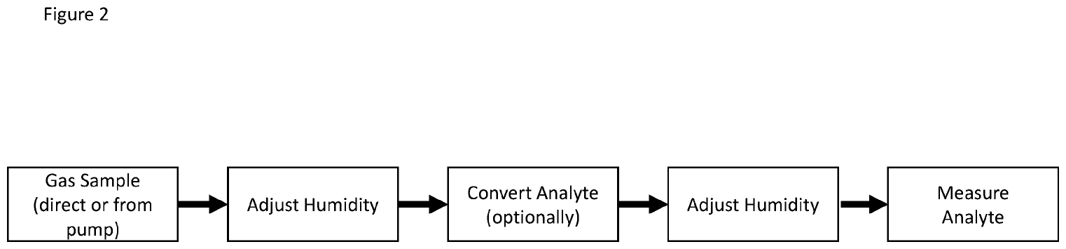

FIG. 2 shows one embodiment of a system or method for determining the concentration of at least one analyte in a gas sample by adjusting humidity, optionally converting at least one analyte, adjusting humidity, and measuring the at least one analyte. In some embodiments, adjusting humidity comprises at least one of dehumidifying; humidifying; and equilibrating to ambient or near ambient relative humidity. In some embodiments, “near ambient relative humidity” means within 50% or less of the relative humidity, within 25% or less of the relative humidity, within 20% or less of the relative humidity, within 15% or less of the relative humidity, within 10% or less of the relative humidity, within 5% or less of the relative humidity, or within 3% or less of the relative humidity. In some embodiments, the analyte is converted by oxidation. In some embodiments, the analyte is converted by reduction. In some embodiments, the analyte is converted by formation of complexes. In some embodiments, the analyte is converted by covalent bonding. In some embodiments, the analyte is converted by chemical reactions. In some embodiments, the analyte is converted by a change in physical state. In some embodiments, the analyte is condensed from a gas into a liquid. In some embodiments, the analyte is condensed from a liquid to a solid. In some embodiments, the analyte forms a plasma. In some embodiments, the analyte volatilizes from a liquid or solid to a gas. In some embodiments, the analyte converts from a solid to a liquid. In another aspect of the technology, the analyte is converted by humidity adjustment. In some embodiments, the analyte is measured by a sensing technology known in the art. In some embodiments, the analyte is measured by sensors as previously described by the applications incorporated above. In some embodiments, the analyte is measured by chemiresistive sensors. In some embodiments, the analyte is measured by metal oxide sensors (MOS, CMOS, etc.). In some embodiments, the analyte is measured by electrochemical sensors. In some embodiments, the analyte is measured by MEMS sensors. In some embodiments, the analyte is measured by acoustic sensors. In some embodiments, the analyte is measured by IR sensors. In some embodiments, the analyte is measured by laser sensors. In some embodiments, the analyte is measured by chemiluminescence. In some embodiments, the analyte is measured by GC/MS sensors. In some embodiments, the analyte is measured by Field Asymmetric Ion Mobility sensors. In some embodiments, the analyte is measured by graphene sensors. In some embodiments, the analyte is measured by electrochemical sensors. In some embodiments, the analyte is measured by optical sensors. In some embodiments, the analyte is measured by FET, MOSFET, and ChemFET sensors. In some embodiments, the analyte is measured by sensors previously described by the authors.

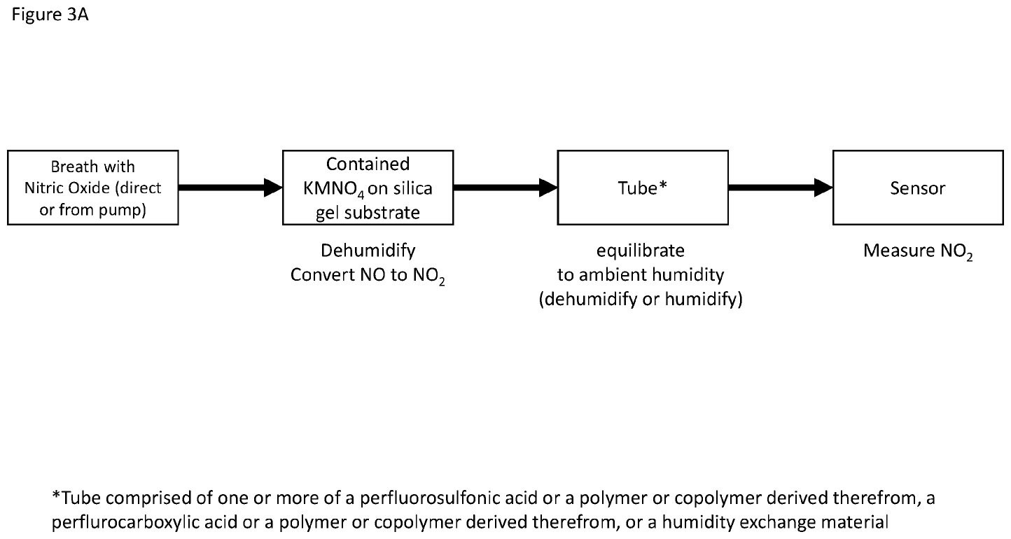

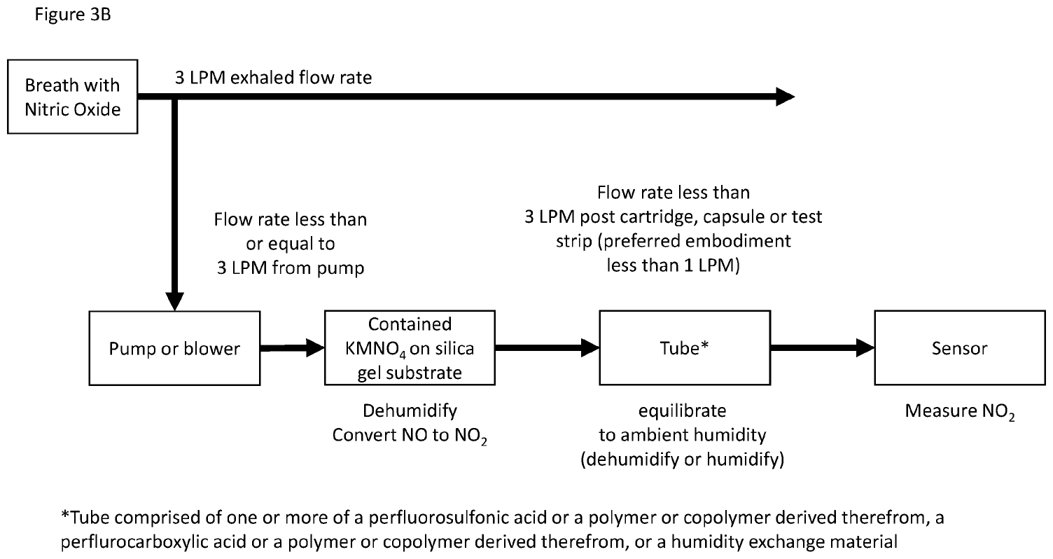

FIG. 3A shows one embodiment of use of a system for determining the concentration of at least one analyte in a gas sample by adjusting humidity using potassium permanganate on a silica gel substrate, adjusting humidity using a tube comprising one or more of a perfluorosulfonic acid or a polymer or copolymer derived therefrom, a perflurocarboxylic acid or a polymer or copolymer derived therefrom, or a humidity exchange material, and measuring the analyte. In one embodiment, a patient's breath, contains nitric oxide, is blown either directly or driven by a pump, fan or blower and flows through a cartridge containing potassium permanganate on a silica gel substrate. Humidity is adjusted by dehumidification and nitric oxide is converted into nitrogen dioxide in a single step. The nitrogen dioxide flows through a tube comprising one or more of a perfluorosulfonic acid or a polymer or copolymer derived therefrom, a perflurocarboxylic acid or a polymer or copolymer derived therefrom, or a humidity exchange material to equilibrate to ambient humidity. In one embodiment, the tube comprising one or more of a perfluorosulfonic acid or a polymer or copolymer derived therefrom, a perflurocarboxylic acid or a polymer or copolymer derived therefrom, or a humidity exchange material dehumidifies the breath. In another embodiment, the tube comprising one or more of a perfluorosulfonic acid or a polymer or copolymer derived therefrom, a perflurocarboxylic acid or a polymer or copolymer derived therefrom, or a humidity exchange material humidifies the breath. In one embodiment, the sensor measures at least one of nitric oxide or nitrogen dioxide.

FIG. 3B shows one embodiment of a system for determining the concentration of at least one analyte in a gas sample wherein the gas sample is moved through the system with the aid of a pump, fan, or blower. In some embodiments, the pump, fan, or blower samples a side stream from a main gas stream. For example, a human or animal exhales at 3 LPM and the pump pulls a side stream of less than 3 LPM. Other flow rates are possible without deviating from the spirit of the technology. In some embodiments, the cartridge, capsule, test strip, or test strip chamber serves a purpose of reducing the flow rate and enabling more efficient humidity adjustment by the tube comprising one or more of a perfluorosulfonic acid or a polymer or copolymer derived therefrom, a perflurocarboxylic acid or a polymer or copolymer derived therefrom, or a humidity exchange material.

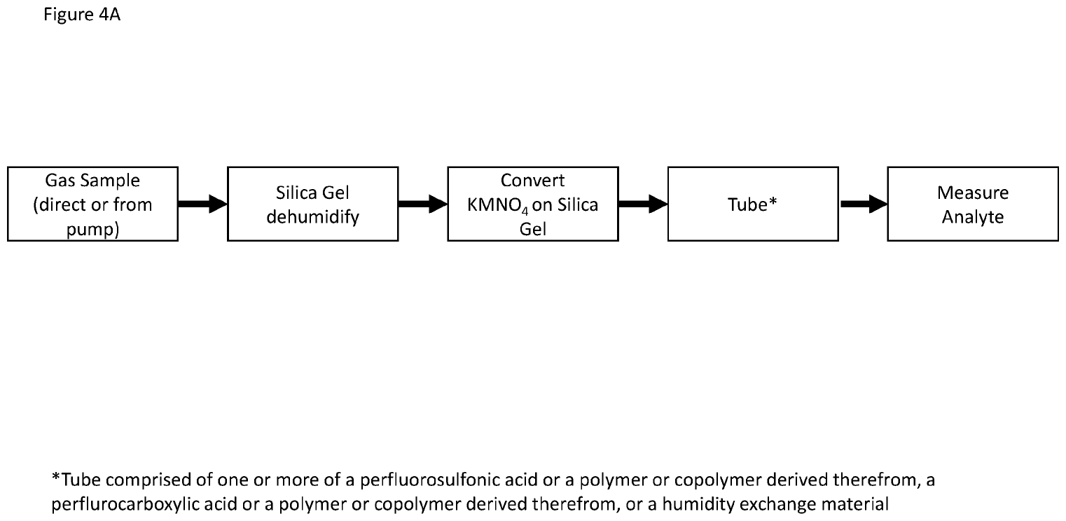

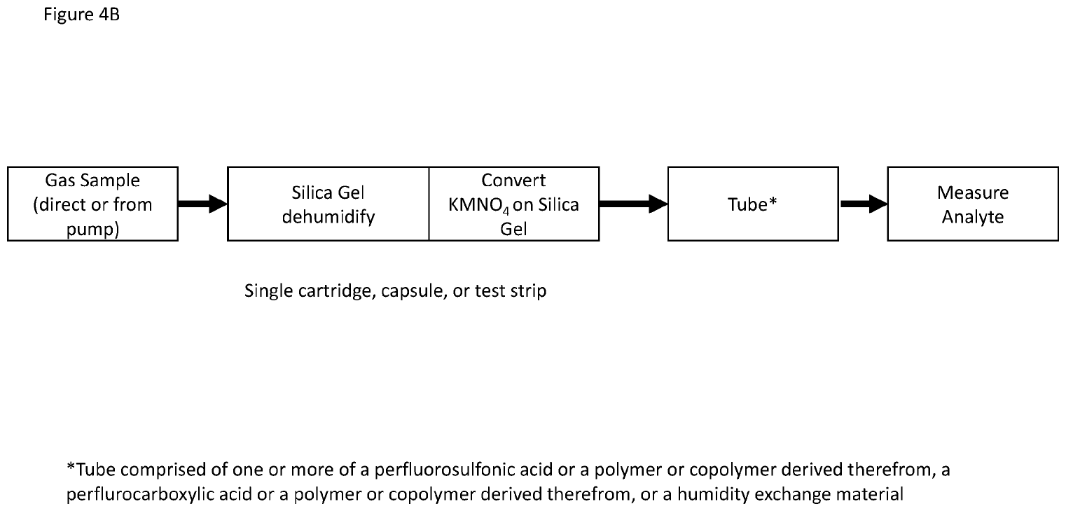

FIGS. 4A and 4B show alternate embodiments of a system for determining the concentration of at least one analyte in a gas sample. In FIGS. 4A and 4B, a gas sample is dehumidified through a silica gel, the analyte is chemically altered using a potassium permanganate on a silica gel substrate, humidity is adjusted through a tube comprising one or more of a perfluorosulfonic acid or a polymer or copolymer derived therefrom, a perflurocarboxylic acid or a polymer or copolymer derived therefrom, or a humidity exchange material, and the analyte is measured by a sensor. In FIG. 4B, a gas sample is dehumidified through a silica gel and the analyte is converted using a potassium permanganate on a silica gel substrate in a single cartridge, capsule, test strip, or test strip chamber. In one embodiment of FIG. 4A or 4B, nitric oxide is converted to nitrogen dioxide which is then measured.

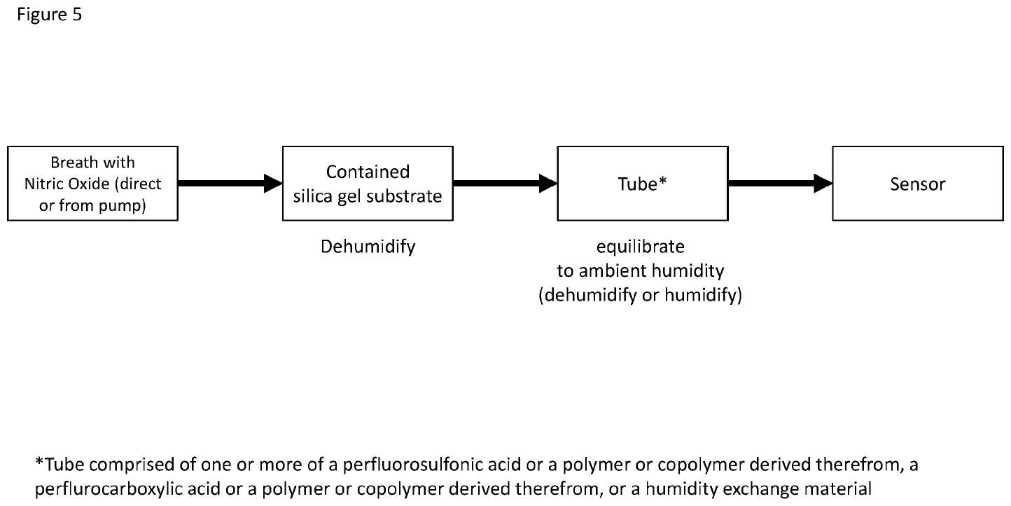

FIG. 5 shows one embodiment of a system for determining the concentration of at least one analyte in a gas sample wherein a patient's breath, containing nitric oxide, is blown either directly or moved with a pump and flows through at least one of a cartridge, capsule or test strip containing a silica gel substrate to dehumidify the breath. The resulting gas sample flows through a tube comprising one or more of a perfluorosulfonic acid or a polymer or copolymer derived therefrom, a perflurocarboxylic acid or a polymer or copolymer derived therefrom, or a humidity exchange material to equilibrate to ambient humidity. In one embodiment, the tube comprising one or more of a perfluorosulfonic acid or a polymer or copolymer derived therefrom, a perflurocarboxylic acid or a polymer or copolymer derived therefrom, or a humidity exchange material dehumidifies the breath. In another embodiment, the tube comprising one or more of a perfluorosulfonic acid or a polymer or copolymer derived therefrom, a perflurocarboxylic acid or a polymer or copolymer derived therefrom, or a humidity exchange material humidifies the breath. In one embodiment, the sensor measures at least one of nitric oxide or nitrogen dioxide.

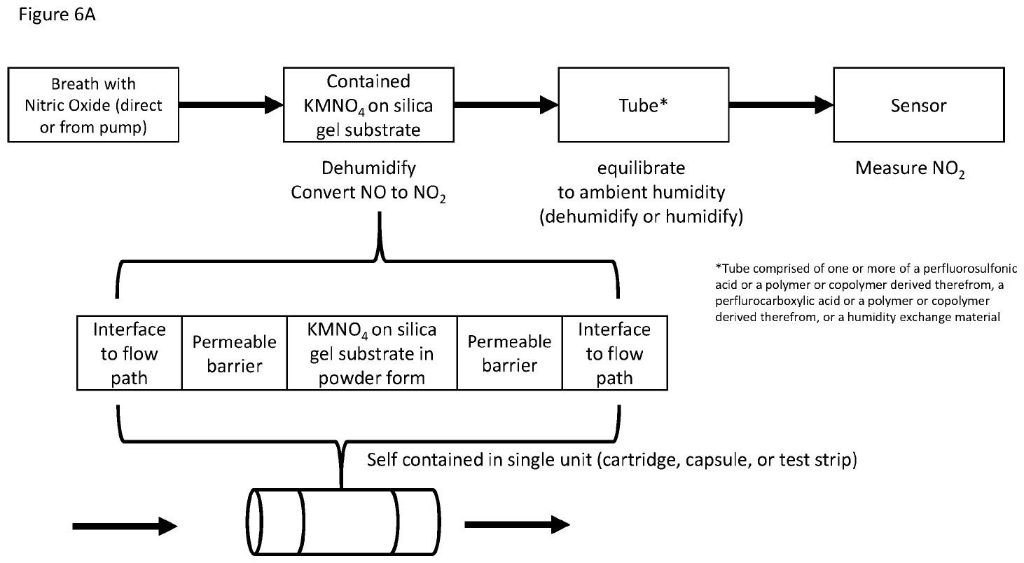

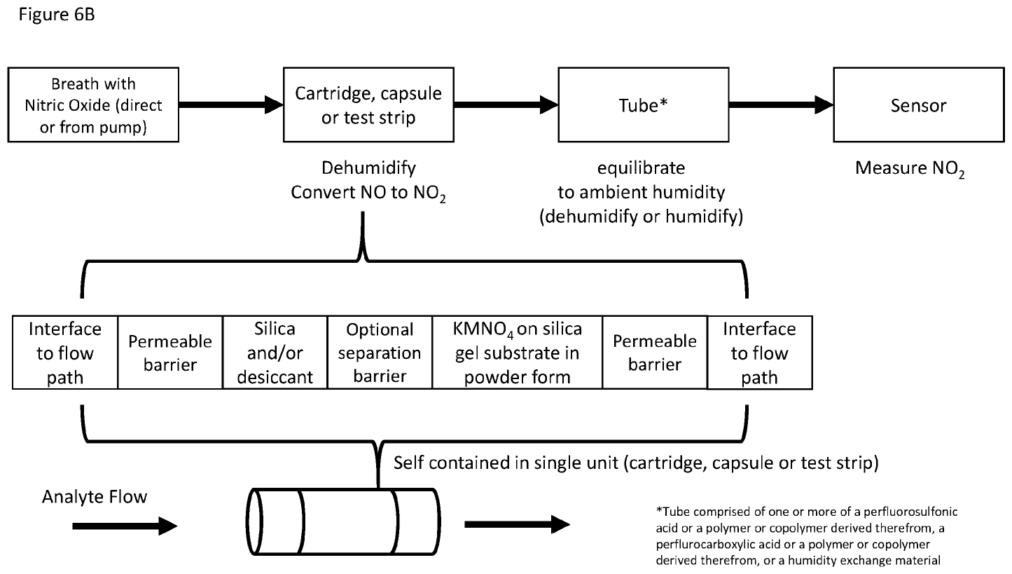

FIG. 6A depicts one example of a cartridge, capsule, test strip, or test strip chamber as described herein. The cartridge, capsule, test strip, or test strip chamber contains an interface to the flow path, a permeable barrier or membrane to contain the functionalized silica gel in powder form and prevent it from escaping the cartridge, capsule, test strip, or test strip chamber while allowing gas to flow through the cartridge, capsule, test strip, or test strip chamber, a functionalized silica gel or other desiccant (in this case KMNO4 on silica), a second permeable barrier or membrane, and a second interface to the flow path. In some embodiments, the device chamber, cartridge, capsule, test strip, or test strip chamber contains an interface with press fit, push to connect, compression fit, luer, barbed, male or female, Yor-lok, flared, quick disconnect, quick turn, socket, flange, threaded, sleeve, o-ring seal, seal, beaded, push-on-barbed, threaded, screw on, grip-lock, locking, solvent welded, thermal welded, and/or bonded with an adhesive. Any other appropriate structure or material known in the art may be used.

FIG. 6B depict an embodiment of a cartridge, capsule, test strip, or test strip chamber. The embodiment contains an interface to the flow path, a permeable barrier or membrane to capture the powder (in this case a silica desiccant) and prevents the powder from escaping the cartridge, capsule or test strip while allowing gas to flow through the cartridge, capsule or test strip, a silica or another desiccant, optionally another permeable barrier or membrane to separate the silica from a second desiccant or functionalized material, a functionalized silica gel or other desiccant (in this case KMNO4 on silica), a second permeable barrier or membrane, and a second interface to the flow path. The interfaces can be any of those described above.

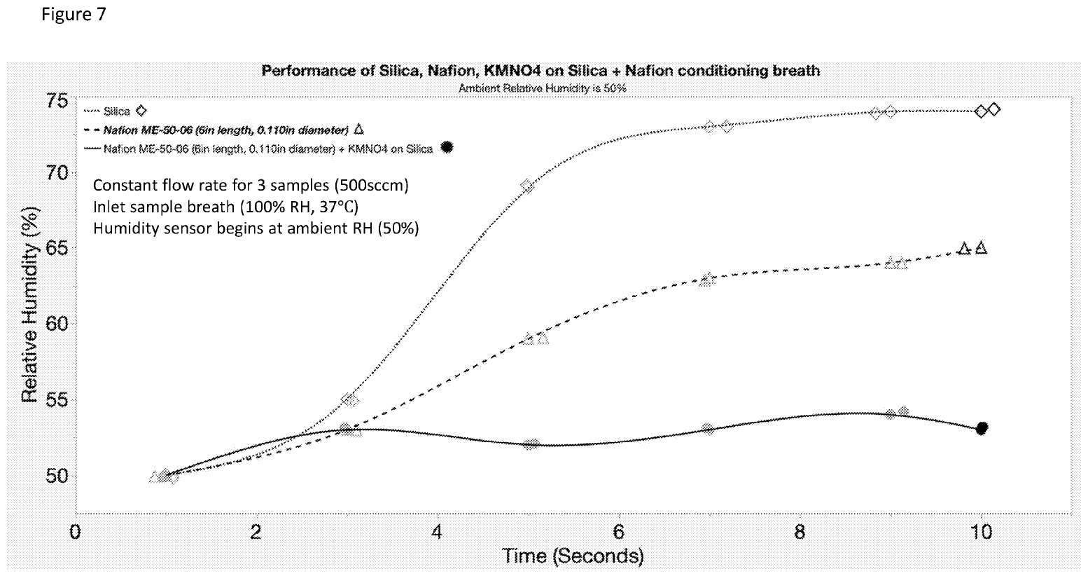

FIG. 7 depicts the performance of one configuration of the technology versus two standard configurations of breath conditioning. The first standard configuration comprises 1 g of silica gel, represented by diamond data points and a dotted line. The second configuration comprises Nafion® tube (ME-50-06 (6 inches in length, 1.07 mm in inner diameter, 1.35 mm outer diameter) from PermaPure, LLC, represented by triangle data points and a dashed line. An embodiment of the present technology comprising one of a cartridge, capsule, test strip, or test strip chamber containing potassium permanganate on silica and a Nafion® tube (ME-50-06 (6 inches in length, 1.07 mm in inner diameter, 1.35 mm outer diameter) from PermaPure, LLC and, represented by circle data points and a solid line. In this embodiment, the system includes conversion/chemical alteration and humidity adjustment as a first step followed by a second step of humidity adjustment. Three separate breath samples are passed through the three separate configurations prior to measurement by a sensor. Inlet breath is 100% relative humidity and 37° C. The patient exhales at 3LPM and a pump siphons a side stream at less than or equal to 3 LPM through the three configurations and relative humidity is monitored at the surface of the sensor. The ambient humidity is 50%. Table 1 demonstrates the performance of the technology in conditioning the gas stream for analysis. The illustrative embodiment of the technology produces a difference in relative humidity of 3% RH between ambient and the sample whereas the silica and Nafion® tube produce a difference of 24% RH and 15% RH respectively as shown in Table 1. In one embodiment, the delta % RH between the sample and the ambient humidity is less than or equal to 20% RH. In another embodiment, the delta % RH between the sample and the ambient humidity is less than or equal to 15% RH. In a further embodiment, the delta % RH between the sample and the ambient humidity is less than or equal to 10% RH. In still other embodiments, the delta % RH between the sample and the ambient humidity is less than or equal to 5% RH. In another embodiment, the delta % RH between the sample and the ambient humidity is less than or equal to 3% RH.

TABLE 1
Comparative performance of the technology as demonstrated
by a configuration in which Silica gel functionalized
with potassium permanganate is positioned proximally
to a Nafion ® tube (ME110-06 PermaPure, LLC).

| | | | Silica gel |
|---|---|---|---|
| | | | functionalized with |
| | | | potassium permanganate |
| | | | positioned proximally |
| | | Nafion ® | to a Nafion ® |
| | | tube (ME05-06 | tube (ME110-06 |
| | Silica | PermaPure, LLC) | PermaPure, LLC). |
| Ambient RH | 50 | 50 | 50 |
| Ending RH | 74 | 65 | 53 |
| after sample | | | |
| exposure | | | |
| Sample Delta | 24 | 15 | 3 |
| RH (ending RH − | | | |
| ambient RH | | | |

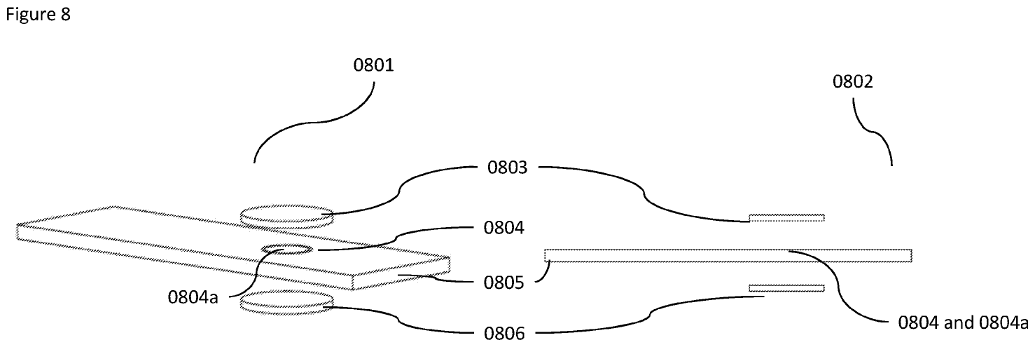

FIG. 8 depicts a non-limiting example of a test strip to condition a gas stream. In this embodiment, the test strip is a combination of flexible layers. Those skilled in the art of diagnostic sensors for blood, urine, and fecal analysis would appreciate the types of materials used. These materials include but are not limited to the materials previously described. The test strip is shown with its layers separated and in two different orientations [0801] and [0802]. It contains two membrane layers [0803] and [0806] and a spacing layer [0805]. The spacing layer [0805] further defines at least one hole [0804]. In some embodiments, the at least one hole is filled with at least one material to condition the gas stream [0804a]. In this embodiment, the membrane layers [0803] and [0806] are larger than the hole [0804] in the spacing layer [0805] and have a sufficient pore diameter to retain any material [0804a] contained in spacing layer [0805]. The gas conditioning materials may be comprising any number of combinations of the materials previously described. The layers of the strip may be bound together by additional layers such as pressure or heat sensitive adhesives. Layers may also be bound together by other techniques such as, thermal bonding, sonic welding, two-part adhesives, moisture cure adhesives, and other techniques know to those in the art. Various configurations are possible such that the gas may pass through each of the layers [0803], [0805], [0806] and through the material to condition the gas stream [0804a].

In one embodiment, the spacing layer [0805] is filled with a powder containing a permanganate salt. In another embodiment, the spacing layer is filled with a permanganate salt on a silica gel, substrate or sphere (e.g. a potassium permanganate functionalized silica gel, a potassium permanganate impregnated silica, a potassium permanganate functionalized silica, a permanganate bound to silica, a permanganate decorated silica, or a permanganate salts adsorbed onto silica). In another embodiment the spacing layer is filled with a reactive or catalytic metal or metal oxide, such as palladium, platinum, or cerium oxide. In another embodiment the spacing layer is filled with a chemical complexing agent. In another embodiment the spacing layer is filled with an oxidizing agent. In another embodiment the spacing layer is filled with a reducing agent. In another embodiment the spacing layer is filled with a molecular sieve to adsorb contaminant species. In another embodiment, the spacing layer is filled with an ion exchange resin. In another embodiment, the spacing layer is filled with a pH modifier. In another embodiment, the spacing layer is filled with a desiccant. In another embodiment, the spacing layer is filled with a humectant. In another embodiment, the spacing layer is filled with a dynamic humidity stabilizer. In another embodiment, the spacing layer is filled with a mixture of compounds to perform multiple reactions. In some embodiments, the test strip [0801] further contains a sensing layer (not shown).

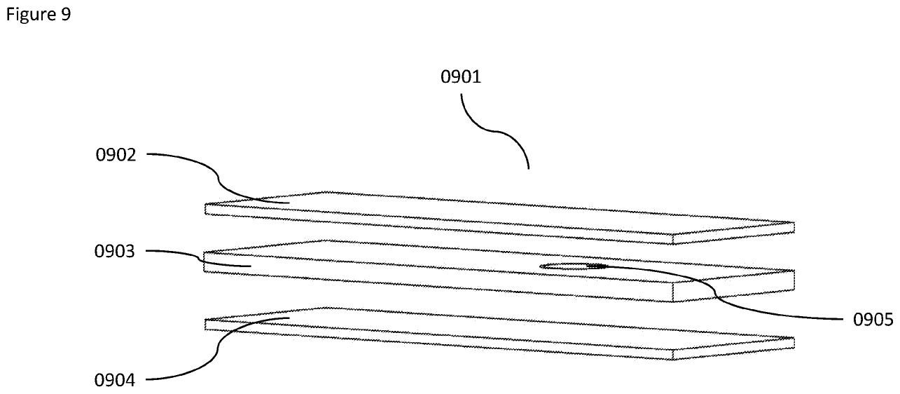

FIG. 9 depicts another configuration of a test strip [0901] for conditioning a gas stream. The configuration is similar to FIG. 8 except the membrane layers [0902] and [0904] cover a larger area of the spacing layer [0903] but still retain any material [0905] contained in the spacing layer [0903]. In some embodiments, the membrane layers [0902] and [0904] have the same dimensions as the spacing layer [0905].

In some embodiments, the layer and membrane combinations described in FIGS. 8 and 9 may be stacked on top of each other any number of times. For example, it is envisioned that the stack may include, in order, a first membrane layer, a first flexible layer, a second membrane layer, a second flexible layer, and a third membrane layer. It in some embodiments multiple membrane layers may be disposed between two flexible layers. For example, it is envisioned that the stack may include, in order, a first membrane layer, a first flexible layer, a second membrane layer, a third membrane layer, a second flexible layer, and a fourth membrane layer. In some embodiments the number of membranes layers is m and the number of flexible layers is n, and m and n are equal. In some embodiments the number of membranes layers is m and the number of flexible layers is n, and m equals n+1. In some embodiments the number of membranes layers is m and the number of flexible layers is n, and m equals n−1.

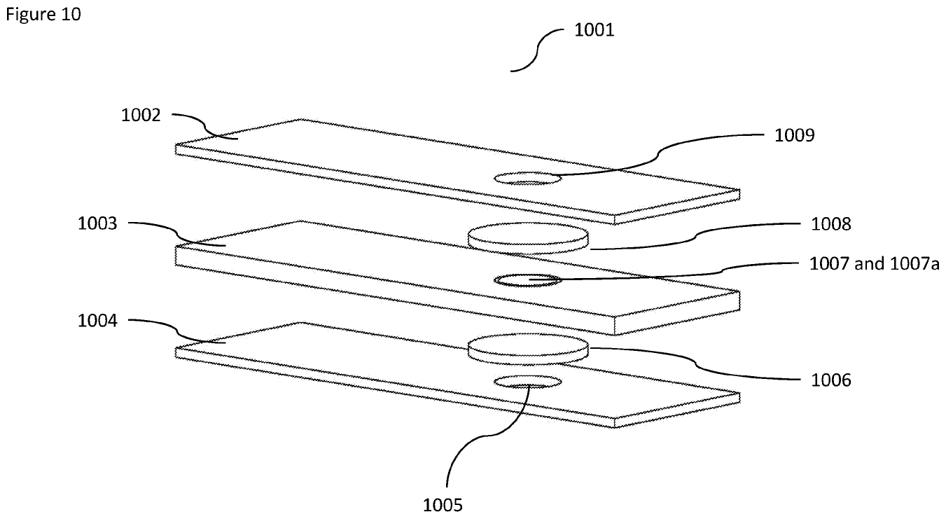

FIG. 10 depicts another embodiment of a test strip for conditioning gas in a sample. The test strip [1001] containing two membrane layers [1008] and [1006], a spacing layer [1003], the spacing layer further containing at least one hole filled with material to condition the gas stream [1007 and 1007a]. Examples of suitable conditioning materials include but is not limited to: a permanganate salt, a permanganate salt on silica gel, a permanganate salt on alumina, a permanganate salt supported on a solid or porous particulate silica gel, silica nanoparticles, palladium powder, desiccants, humectants, dynamic humidity stabilizers, catalytic metals and metal oxides, reducing agents, oxidizing agents, complexing agents, ion exchange resins, pH modifiers, or other chemically active species known in the art for converting or changing chemical species or combinations thereof. The test strip [1001] further at least one protective layer [1002] or [1004], the at least one protective layer [1002] or [1004] further defines at least one hole [1009, 1005] to allow the sample to enter or exit the test strip. The protective layers [1004] and [1002] and membrane layers [1008] and [1006] are bonded or adhered to using previously described methods. In some embodiments the layer holes are in fluid communication such that the sample may pass through the test strip.

In some embodiments, the protective layers [1002] and [1004] are porous membranes. In some embodiments, only the top protective layer [1002] is present. In some embodiments, only the bottom protective layer [1004] is present. In some embodiments, the protective layers don't contain a hole but are sufficiently permeable to enable the gas to pass to the next layer. In some embodiments, the test strip [1001] further contains a sensing layer [not shown].

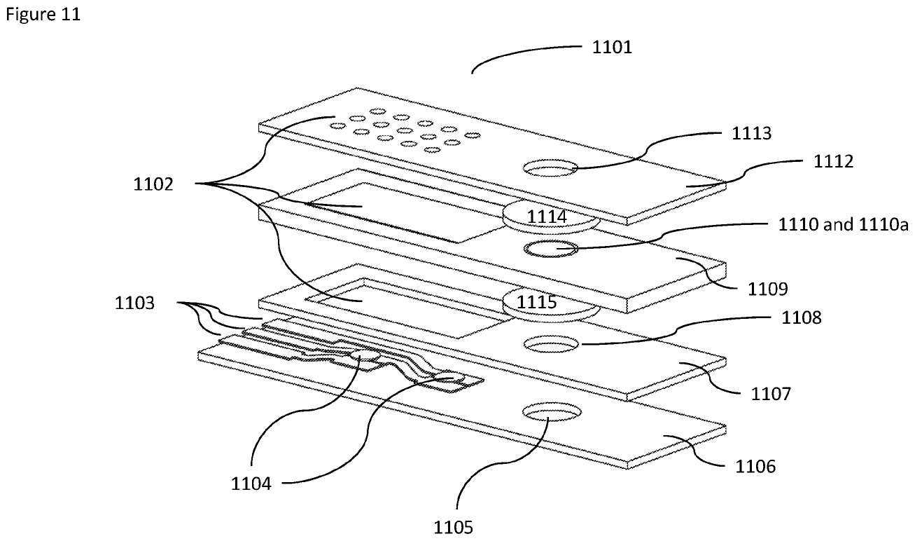

FIG. 11 depicts another embodiment of a test strip for conditioning gas in a sample. The test strip [1101] comprising a first protective layer [1112], a second membrane layer [1114], a third spacing layer [1109], a fourth membrane layer [1115] a fifth spacing layer [1107], and sixth sensing layer [1106]. The spacing layer [1109] further comprising at least one hole filled with material to condition the gas stream [1110 and 1110a]. Examples of suitable conditioning materials include: potassium permanganate, sodium permanganate, a permanganate salt, a permanganate salt on silica gel, a permanganate salt on alumina, a permanganate salt supported on a solid or porous particulate silica gel, silica nanoparticles, palladium powder, desiccants, humectants, dynamic humidity stabilizers, catalytic metals and metal oxides, reducing agents, oxidizing agents, complexing agents, ion exchange resins, pH modifiers, or other chemically active species known in the art for converting or changing chemical species or combinations thereof. The protective layer [1112], second spacing layer [1107], and sensing layer [1106] further defines at least one hole [1113], [1108], [1105] configured to enable gas to traverse the protective layer, second spacing layer, and sensing layer, and providing the gas fluid communication to the test strip. The sensing layer further contains at least one electrode [1103] and at least one sensing chemistry [1104]. The protective layer [1112], first spacing layer [1109], second spacing layer [1107] further contains at least one hole [1102] to enable fluid communication with the first spacing layer [1109] and sensing chemistry [1104]. In another embodiment, the fifth spacing layer [1107] is not present.

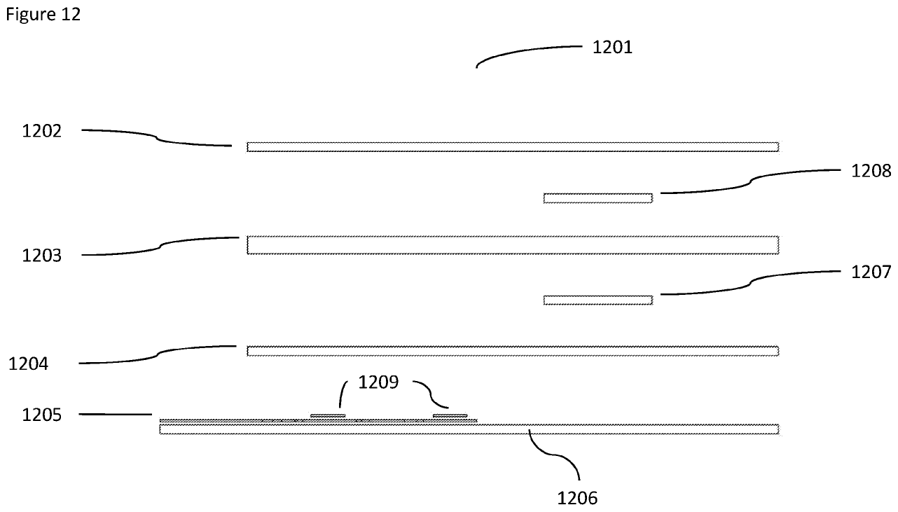

FIG. 12 depicts another embodiment of a test strip for conditioning gas in a sample. The test strip [1201] contains a first protective layer [1202], a second membrane layer [1208], a third spacing layer [1203], a fourth membrane layer [1207], a fifth spacing layer [1204], and sixth sensing layer [1206]. The sensing layer [1206] further contains electrodes [1205], and at least one sensing chemistry [1209]. The spacing layers [1202, 1203, and 1204] further defines at least one hole. The at least one hole of the spacing layer [1203] contains material to condition the gas stream. Examples of suitable conditioning materials may include but is not limited to: a permanganate salt, a permanganate salt on silica gel, a permanganate salt on alumina, a permanganate salt supported on a solid or porous particulate silica gel, silica nanoparticles, palladium powder, desiccants, humectants, dynamic humidity stabilizers, catalytic metals and metal oxides, reducing agents, oxidizing agents, complexing agents, ion exchange resins, pH modifiers, and other chemically active species known in the art for converting or changing chemical species or combinations thereof. The protective layer [1202], first spacing layer [1208], second spacing layer [1204] further defining at least one hole to enable fluid communication of the conditioned gas and the sensing chemistry [1209]. In another embodiment, the spacing layer [1204] is not present.

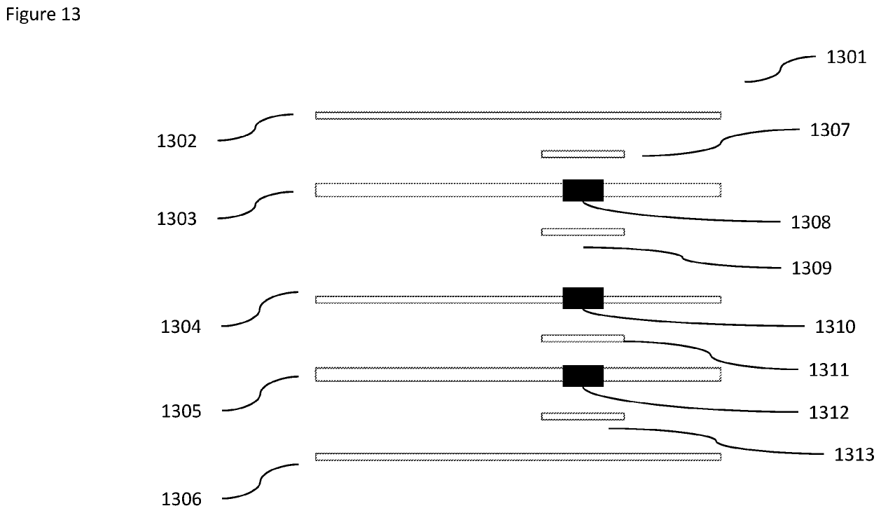

FIG. 13 depicts an embodiment of a test strip for conditioning gas in a sample. The test strip [1301] comprising a first protective layer [1302], a first membrane layer [1307], a first spacing layer [1303], a second membrane layer [1309], a second spacing layer [1304], a third membrane layer [1311], an nth spacer layer [1305], an nth membrane layer [1312], and a optionally a sensing layer[1306]. The sensing layer [1306] further contains electrodes, and at least one sensing chemistry. The spacing layers [1303, 1304, and 1305], up to any number of nth spacing layers, nth membrane layers, or n−1 membrane layers, further comprising at least one hole wherein the at least one hole of each of the layers layer [1303, 1304, and 1305] contains at least one of the same or different conditioning materials in any order to condition the gas stream. Examples of conditioning materials include: a permanganate salt, a permanganate salt on silica gel, silica gel, palladium powder, desiccants, humectants, dynamic humidity stabilizers, catalytic metals and metal oxides, reducing agents, oxidizing agents, complexing agents, ion exchange resins, pH modifiers, and other chemically active species known in the art for converting or changing chemical species or combinations thereof. In some embodiments the conditioning materials are arranged with [1308] contains at least potassium permanganate, [1310] contains at least silica gel, and [1312] contains at least sodium hexametaphosphate. In some embodiments the conditioning materials are arranged with [1308] contains at least potassium permanganate on silica gel, [1310] contains at least silica gel, and [1312] contains at least sodium hexametaphosphate. In some embodiments, the conditioning materials are arranged with [1308] contains at least silica, [1310] contains at least one of a permanganate salt or a permanganate salt on silica, and [1312] contains at least silica. In some embodiments, the conditioning materials are arranged with [1308] contains at least silica, [1310] contains at least one of a permanganate salt or a permanganate salt on silica, and [1312 and 1313] is not present. In some embodiments, the conditioning materials are arranged with [1308] contains at least one of a permanganate salt or a permanganate salt on silica, and [1310] contains at least silica, and [1312 and 1313] is not present. The protective layer [1302], the spacing layers [1303, 1304, and 1305], and sensing layer [1306] further defines at least one hole to enable gas to pass through the test strip. The protective layer [1302], first spacing layer [1303], second spacing layer [1304], and the nth spacing layer [1305], each further defining at least one hole to enable fluid communication of the conditioned gas and the sensing chemistry (not shown).

In another embodiment, the test strip only contains two internal spacing layers [1303] and [1304], three membrane layers [1307], [1309], and [1311] and optionally at least one protective layer [1302] and optionally one sensor [1306]. The spacing layers [1303] and [1304] further contains a material to condition the gas stream as previously described. In one embodiment the materials in spacing layer [1303] contains one of a permanganate salt or a permanganate salt on silica (e.g. functionalized silica) and the materials in spacing layer [1304] contains a desiccant material such as silica. In another embodiment the materials in spacing layer [1303] contains a desiccant material such as silica and the materials in spacing layer [1304] contains one of a permanganate salt or a permanganate salt on silica (e.g. functionalized silica).

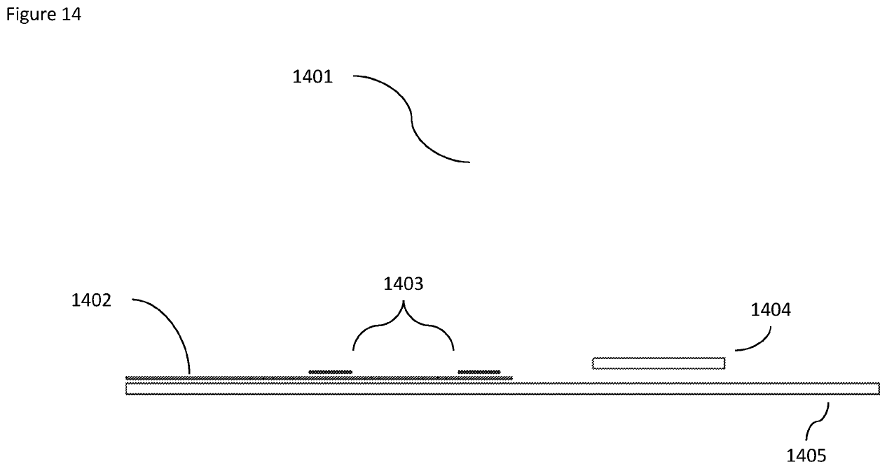

FIG. 14 depicts another embodiment of a test strip for conditioning gas in a sample. The test strip [1401] is configured such that the assembled layers [1404] do not overlap the sensing chemistry [1403], and where the assembled layers [1404] are of any the configurations described within this document. In some embodiments, the configuration layers have at least two membranes, and at least one spacing layer. The spacing layer(s) of [1404] further define a hole, and materials suitable for conditioning the gas as exemplified by above figures are disposed within the hole. The test strip [1401] further contains a sensing layer [1405]. The sensing layer further comprises electrodes [1402], and at least one sensing chemistry [1403]. The layers [1404] and sensing layer [1405] further define at least one hole configured to enable gas to pass through the test strip.

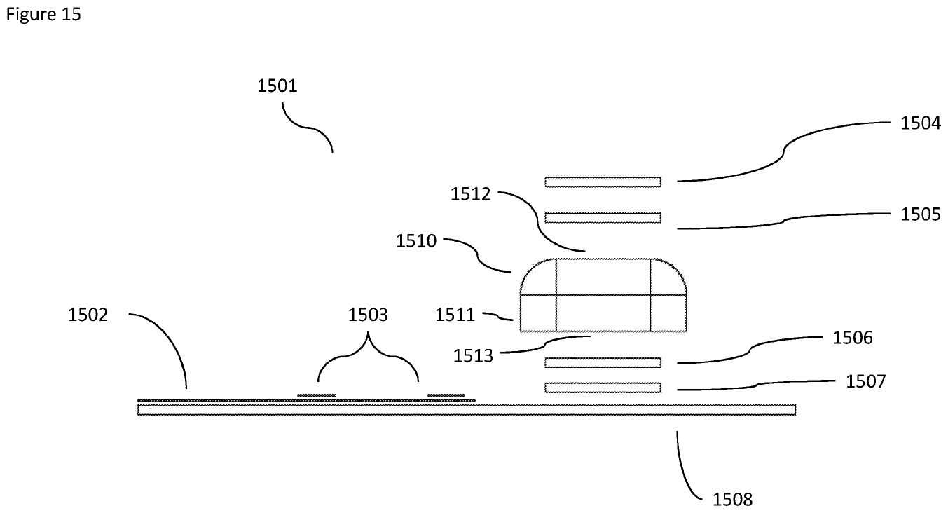

FIG. 15 depicts another embodiment of a test strip [1501] for conditioning gas in a sample using a chamber [1510] configured on a test strip. The chamber [1510] contains the previously described materials used to condition the gas stream and/or at least one of filters, frits or membranes. In some embodiments, the chamber is functionally equivalent to the cartridge, capsule or test strip previously described. In some embodiments, the chamber is hollow. The chamber [1510] may be squared, beveled, or angled. In some embodiments, the chamber comprises at least one of ABS, acrylics, epoxies, metalized plastic, metallized polymers, polycarbonates, polyesters, polyethylene, polypropylene, polystyrene, polystyrene copolymers, polyvinylchloride, silicones, thermoplastics, thermoset polymers or other materials known in the art. This is not intended to be an exhaustive list. In one embodiment, the chamber is at least one of a homogeneous, tri-laminated polystyrene and polycarbonate. The chamber [1510] contains of any number of configurations described within this application for chambers, cartridges, test strips, or capsules suitable to house at least one material to condition the gas stream. In some embodiments, the chamber [1510] further defines at least one hole, opening, slot, or open surface. In one embodiment, the chamber contains of at least one membrane. In one embodiment, the chamber comprises a first and a second membrane. In some embodiments, material to condition the gas stream is contained between the first and second membrane. In some embodiments, the material to condition the test strip is contained by the at least one membrane. The membrane selected with sufficient pore size to encapsulate any contained material. The test strip [1501] further optionally contains a sensing layer [1508]. The sensor substrate layer further contains electrodes [1502], at least one sensing chemistry [1503]. In some embodiments, the sensing layer [1508] further defines at least one hole. The at least one chamber hole may be at least one of an inlet or an outlet. In some embodiments, the at least one hole may be on any surface of the chamber such that the gas may pass through the conditioning material. Examples of hole locations include but is not limited to [1511], [1512], 1513]. The test strip [1501] further contains at least one top [1504] or bottom [1507] protective layers and at least one membrane layers [1505, and 1506]. The top [1504] or bottom [1507] layer further defines at least one hole. In some embodiments, the chamber [1510] is bonded or adhered to at least one of a membrane, flexible layer, or sensing layer. The chamber may be bonded or adherence using techniques previously described for the chamber, capsule or test strip.

In some embodiments, the chamber [1510] containing the material to condition the gas stream is tapered. Various degrees of taper are possible without deviating from the spirit of the technology. Tapering the chamber enables more efficient filling of the chamber with a material to condition the gas stream during manufacturing. Any chamber of any of the provided examples or embodiments of the technology may be tapered.

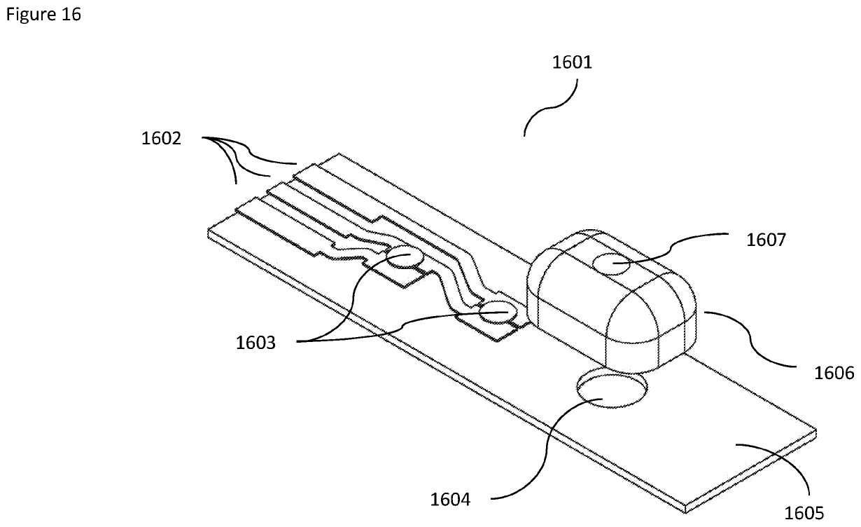

FIG. 16 depicts another embodiment of a test strip for conditioning gas in a sample comprising of a chamber on a test strip. The test strip [1601] comprises a chamber [1606], and a sensing layer [1605]. The chamber further defines at least one hole. The at least one chamber hole may be at least one of an inlet or an outlet. The sensing layer further comprises of at least one electrode [1602] and at least one sensing chemistry [1603]. In some embodiments, the sensing layer [1605] furthering defines at least one hole [1604]. In some embodiments, the at least one hole in the sensing layer [1604] is configured to enable fluid communication between the at least one chamber hole defining a chamber inlet [1607]. In some embodiments, the chamber further comprises at least one of a membrane, filter, or frit positioned within the chamber. In some embodiments, the chamber [1606] contains at least one of a material to condition the gas stream. The materials of the chamber may be comprising those previously described for a cartridge, capsule, test strip, or test strip chamber. The configurations of the chamber may be the same or similar to those previously described for a cartridge, capsule, test strip, or test strip chamber. In some embodiments, the chamber [1606] is bonded or adhered to at least one of a membrane, flexible layer, or sensing layer. The chamber may be bonded or adherence using techniques previously described for the chamber, capsule or test strip. In some embodiments the chamber inlet [1607] is in fluid communication with a tube. In some embodiments, at least one of the chamber outlet or at least one hole [1604] in the sensing layer [1605] is in fluid communication with a tube.

In one embodiment, the sensing layer [1605] further defines a hole [1604] to enable fluid communication between the chamber inlet [1607], at least one sensing chemistry [1603], and optionally sensor electrodes [1602]. In one embodiment, there is at least one of a membrane, filter or frit between the chamber [1606] and the sensing layer [1605] that covers, overlaps, or overlays the at least one sensing layer hole [1604]. In one embodiment, there is at least one of a membrane, filter, or frit contained within the chamber wherein the at least one membrane, filter or frit, covers, overlaps, or overlays the a least one hole defining a chamber inlet [1607]. In some embodiments, the membrane is sufficiently porous to capture the conversion material in the chamber [1606] while still enabling gas to pass through it. In one embodiment, the at least one membrane dimensions are at least the same as the dimensions of the bottom of the chamber [1606]. In one embodiment, the length and width or diameter of the membrane is greater than the diameter of the hole [1604] in the sensing layer [1605]. In one embodiment, the chamber [1606] contains an inlet [1607] to enable gas to enter. In some embodiments, the sensing layer [1605] is not present.

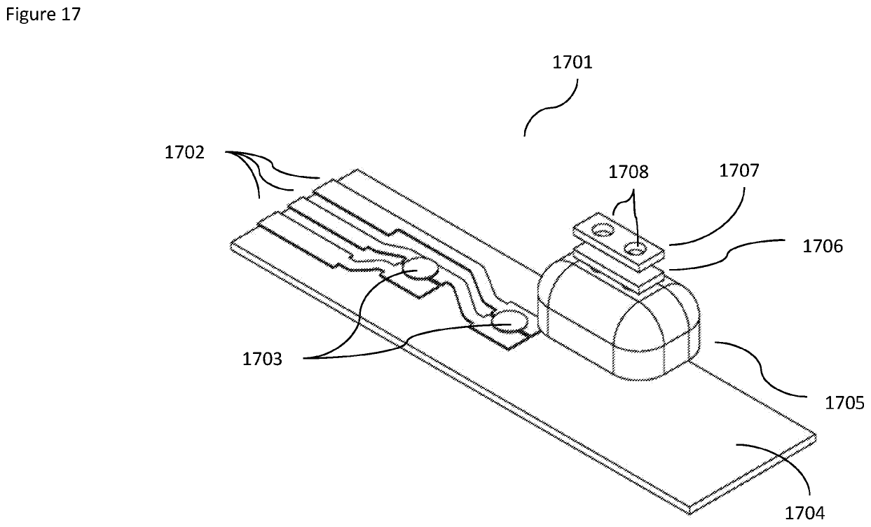

FIG. 17 demonstrates another embodiment of a test strip for conditioning gas in a sample using a chamber [1705] configured to house at least one of a membrane, filter or frit and at least one of a material to condition the gas sample. In this embodiment, the chamber contains a membrane [1706] that is positioned either internally or externally on the chamber [1705]. The membrane has sufficient porosity to encapsulate the material to condition the gas sample, while enabling gas to pass through it and into the chamber [1705]. In one embodiment, the chamber [1705] also contains at least one protective layer [1707] further defines at least one hole. In one embodiment, the at least one protective layer [1707] contains at least one hole which is one or more of an inlet or an outlet [1708] to enable the gas to enter and exit the chamber [1705]. In this example, the side of the chamber [1705] opposite of the protective layer [1708] is sealed so that the gas may only enter and exit through the holes [1708] in the protective layer [1707].

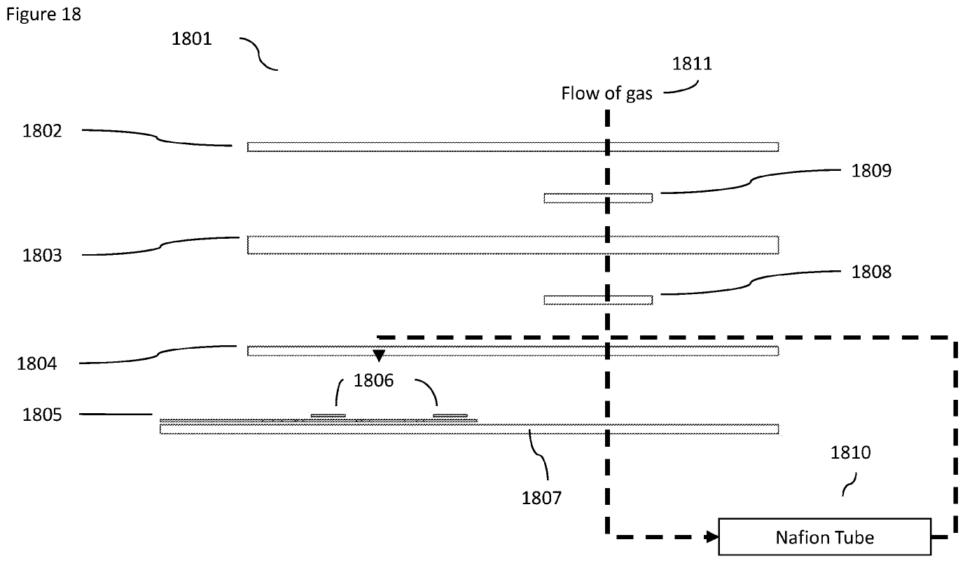

FIG. 18 depicts the flow of gas through the system for condition a gas sample. In this embodiment, gas is passed through the test strip [1801] layers [1802, 1803, 1804, 1807], through the tube [1810], and back through the test strip layers [1802, 1803, 1804,] to the at least one sensing chemistry [1806]. In one embodiment, the test strip [1801] comprises a first protective layer [1802], a second membrane layer [1809], a third spacing layer [1803], a fourth membrane layer [1808], a fifth spacing layer [1804], and sixth sensing layer [1807]. The sensing layer [1807] further comprise at least one electrode [1805], and at least one sensing chemistry [1806]. The protective layer [1802], and the spacing layers [1803, and 1804] further define at least one hole, and with at least one of the at least one hole of the spacing layer [1803] filled with a material to condition the gas stream as described previously. The protective layer [1802] and spacing layers [1803 and 1804] further defines at least one first hole to enable fluid communication between the gas sample [1811], test strip [1801] and tube [1810]. The protective layer [1802], and the spacing layers [1803, and 1804] further defines at least one second hole to enable fluid communication between the tube [1810] and the sensing chemistry [1806]. In this embodiment, gas passes through the test strip, to a tube, and back through the test strip as shown by the dotted arrow. The same flow path is possible in the various sensor configurations depicted in any of the figures or described in any of the examples or elsewhere in the description. In one embodiment, layer [1804] is not present. In another embodiment layer [1802] is not present.

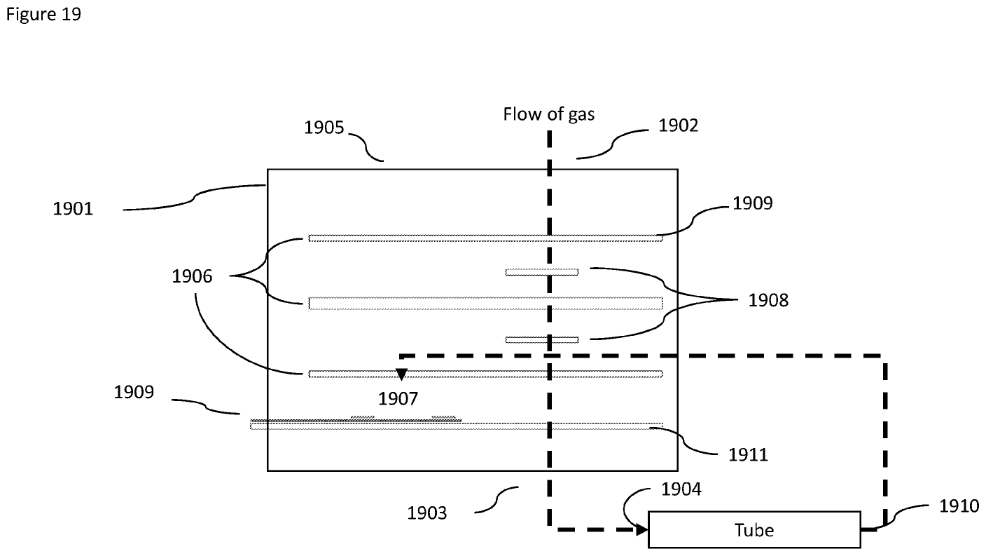

FIG. 19 depicts another embodiment of a test strip that is similar to FIG. 18, wherein the test strip is housed within a device chamber [1901] wherein the device chamber is configured to have at least one first inlet [1902], optionally at least one second inlet [1905] and optionally least one outlet [1903]. In some embodiments, the device chamber is not fully enclosed. The device chamber is further configured to interface with the test strip top [1909] and bottom [1908] layers such that gas may flow into the device chamber inlet [1902] through the layers of the test strip [1906], [1908] and [1911] and back thru the device chamber outlet [1903]. The first device chamber outlet [1903] is further configured to be in fluid communication with the inlet of at least one tube [1904]. The second device chamber inlet [1905] is further configured to be in fluid communication with the outlet of the at least one tube [1910]. In some embodiments, the second device chamber inlet [1905] interfaces with at least one of the test strip layers.

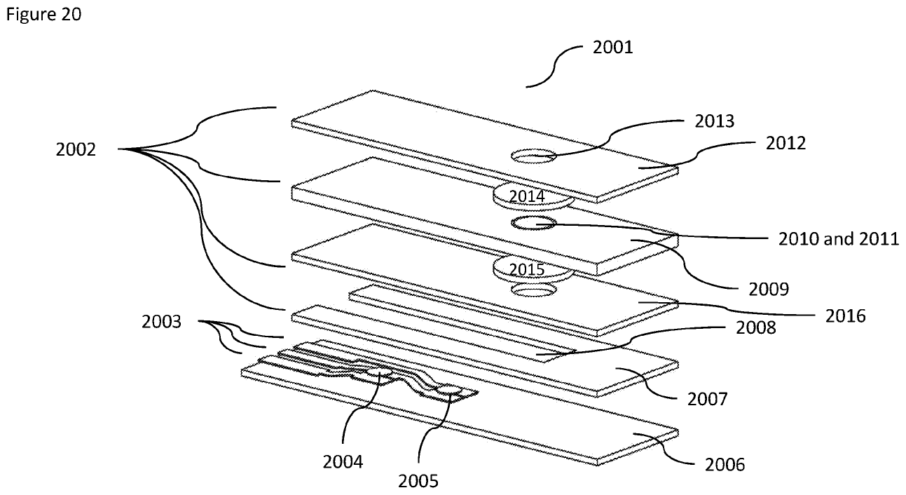

FIG. 20 depicts another embodiment of a gas conditioning system comprising a test strip wherein at least one of the test strip layers [2007] further contains a channel [2008] wherein the channel is in fluid communication with a sensor or at least one sensing chemistry. In one embodiment, the test strip [2001] comprises a first protective layer [2012], a first membrane layer [2014], a first spacing layer [2009], a second membrane layer [2015], optionally a second spacing layer [2016], a channel layer [2007], and optionally a sensing layer [2006]. The first protective layer [2012] defining at least one hole [2013] to enable the gas to enter the test strip. The first spacing layer [2009] further defines at least one hole wherein the at least one hole is filled with material to condition the gas stream [2010, 2011]. The first membrane layer [2014] is configured to overlay at least one side of the at least one hole [2010, 2011] in the first spacing layer [2009]. The second membrane layer [2015] is configured to overlay at least one side of the at least one hole [2010, 2011] in the first spacing layer [2009]. The optional spacing layer [2016] defines of at least one hole. The sensing layer [2006] further comprises of at least one electrode [2003] and at least one sensing chemistry [2004, 2005]. The sensing layer optionally comprising at least one hole. In some embodiments, the sensing layer is replaced by a second protective layer. The second protective layer optionally defines at least one hole. The channel layer [2007] further defines a channel [2008] in fluid communication with the sensing chemistry and any one of the at least one holes in the previously described layers. In one embodiment, the channel [2008] in the channel layer [2007] is in fluid communication with the sensor or sensing chemistry and the flow path of gas through the test strip. In one embodiment, the channel [2008] in the channel layer [2007] is open on at least one end to enable the gas to escape the test strip. In one embodiment, the at least one channel [2008] directs the flow of gas to at least one sensing chemistry [2004] and/or [2005] or other type of sensor. In one embodiment of FIG. 20, gas flows into the test strip via [2013], through layers [2012, 2009, 2016] and through the membrane layers [2014, 2015] and is directed by the channel [2008] in the channel layer [2007] to the at least one sensing chemistry [2004 or 2005] and exits the test strip. In the shown embodiment, the gas exits near the electrodes [2003] but other exit paths are possible without deviating from the spirit of the technology. In one embodiment, the channel layer [2007] defines a channel [2008] that enables fluid communication for the gas to the one or more sensors or one or more sensors subsequent to the gas traversing the first spacing layer [2009] hole filled with material to condition the gas stream. In one embodiment, the material is one or more of the permanganate salt, the silica, the permanganate salt on silica, or the activated carbon of the test strip. In one embodiment, layer [2016] is not present.

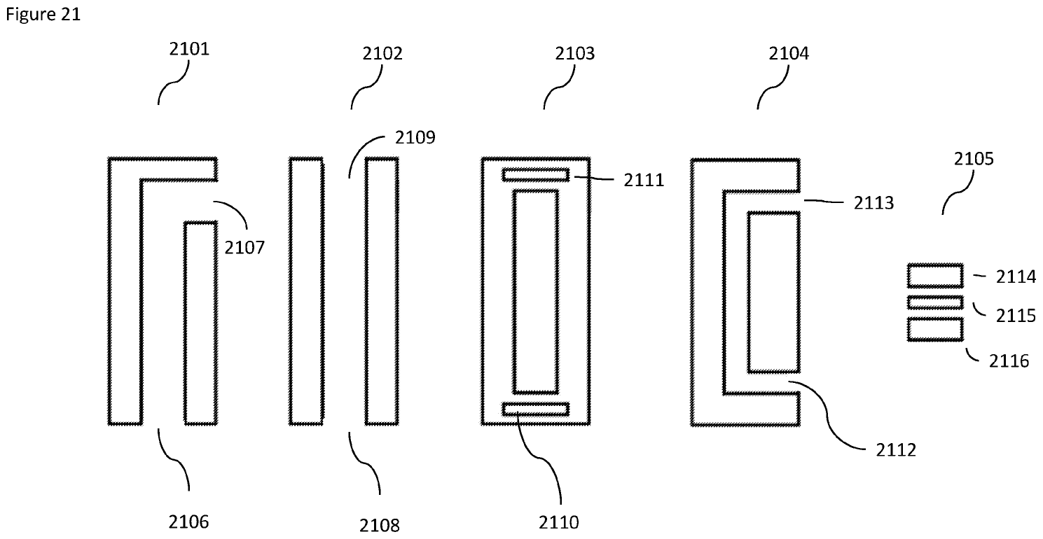

FIG. 21 demonstrates the top view of various configurations of the at least one inlet hole and at least one outlet hole of a conversion cartridge, capsule, test strip, or test strip chamber. In these configurations [2101, 2102, 2103, 2104] the cartridges, capsules, test strip, or test strip chamber may be cylindrical, square, rectangular, or other geometric shapes and profiles. The holes may be on any surface or side of the cartridges, capsules, test strip, or test strip. The configurations define of at least one inlet [2106, 2108, 2110, 2112], and further define at least one outlet [2107, 2109, 2111, 2113] in fluid communication with the inlet [2106, 2108, 2110, 2112]. The inlet and outlet positions may be interchangeable (e.g. [2107] may instead be an inlet, and [2106] may instead be an outlet). In some embodiments, the inlet and outlet are the same. In one embodiment [2103], cartridge, capsule, test strip, or test strip chamber defines at least one hole in at least one part of the side of the cartridge, capsule, test strip, or test strip chamber. In another embodiment the at least one hole serves as the inlet [2110], or outlet [2111]. The configurations [2101, 2102, 2103, and 2104], may contain at least one of a membrane, filter, or frit [2114 and 2116], and at least one material to condition the gas stream [2115] as described previously. These membranes, filters, or frits may be disposed in proximity to one another or separated by one or more non-membrane, non-filter, or non-frit. In some embodiments, the at least one membrane, filter or frit and the at least one material to condition the gas are in the fluid path between the inlet [2106, 2108, 2110, and 2112], and the outlet [2107, 2109, 2111, 2113].

In one embodiment the cartridge interfaces with at least one of a device or a device chamber. In another embodiment the cartridge, capsule, test strip, or test strip chamber interfaces with at least a test strip. In another embodiment the cartridge, capsule, test strip, or test strip chamber interfaces with at least one of a device, a device chamber, and a test strip. In one embodiment, the cartridge, capsule, test strip, or test strip chamber interfaces with a sensor. In another embodiment the cartridge, capsule, test strip, or test strip chamber interfaces with a metal oxide sensing chemistry. In another embodiment the cartridge interfaces with an electrochemical sensing chemistry. In some embodiments, the interface provides fluid communication between the sensor and the gas sample. In some embodiments, the cartridge is adhered or bonded to the sensor or test strip using previously described methods.

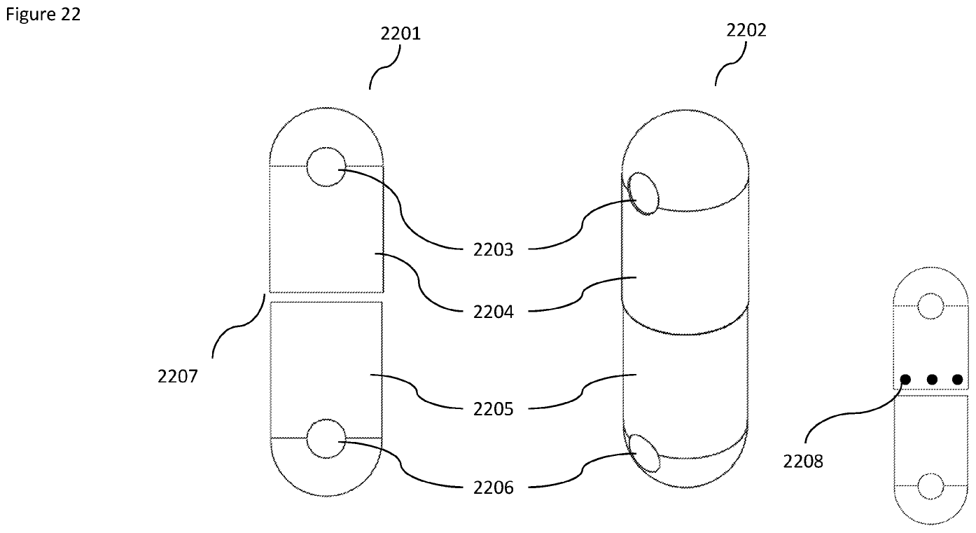

FIG. 22 depicts one embodiment of a capsule to condition a gas, showing the front view [2201] and a perspective view [2202] of the capsule. The embodiment comprises two separate components, a cap [2204] and a body [2205]. The front view shows the cap [2204] and body [2205] as separated components [2207]. In one embodiment, the cap [2204] has a slightly larger diameter than the body [2205] to allow for the body [2205] to slide into the cap [2204], allowing the cap and body to be press fit together. Moreover, the cap [2204] and the body [2205] are hollow to allow for additional components to be placed inside to condition the gas sample and to enable fluid communication between sample inlets [2203] and outlets [2206]. In one embodiment, at least one of the cap [2204] and the body [2205] have additional holes [2208] to enable air to be released from the chamber when press fit during assembly. In one embodiment, the additional holes [2208] are placed near the open edge [2207] of the cap [2204] so that they are sealed, covered, or occluded by the body [2205] when press fit together. In one embodiment, the additional holes [2208] are placed near the open edge [2207] of the body [2205] so that they are sealed, covered, or occluded by the cap [2204] when press fit together.

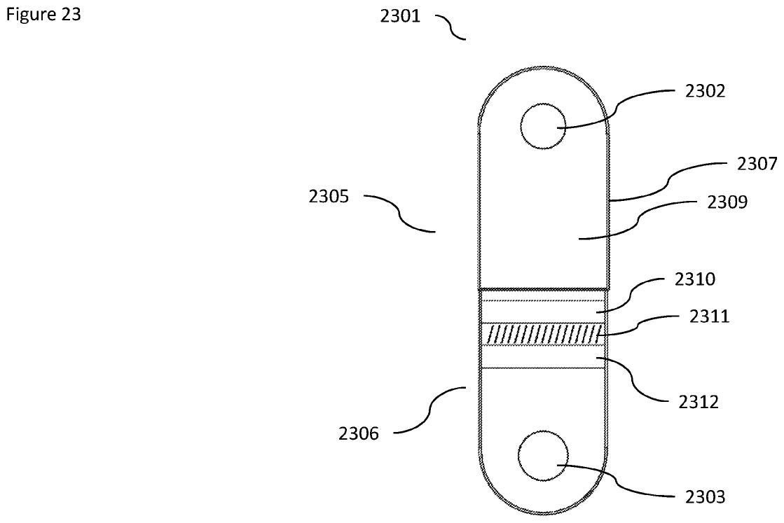

FIG. 23 depicts one embodiment of a cartridge or capsule [2301] to condition a gas. This embodiment demonstrates an assembled capsule or cartridge [2301] containing materials to condition a gas stream, including but not limited to membranes, filters, frits, and conditioning materials as described previously. The capsule comprises a cap [2305] and a body [2306]. The cap [2305] and body [2306] further defining at least one gas inlet [2302] and at least one gas outlet [2303] in fluid communication through the capsule. The inlet [2302] and the outlet [2303] are interchangeable and may be oriented in any configuration as described in FIG. 21. The cap [2305] further comprises an outer wall [2307], and a hollow recessed, inner body [2309]. The cap [2305] or body [2306] is further comprise at least one of a membrane, filter, and/or a frit [2310], at least one of a material to condition the gas sample as previously described [2311], and at least one of a second membrane, filter, and/or frit [2312]. In a preferred embodiment, the at least one material to condition the gas stream is a permanganate salt, or a permanganate salt on silica gel (e.g. functionalized silica gel, sphere, bead nanoparticle). In some embodiments at least one of the at least one of a membrane, filter or frit ([2310] and [2312]) may also condition the sample. Conditioning methods include but are not limited to: oxidizing, reducing, humidifying, dehumidifying, equilibrating with ambient conditions, heating, cooling, chemically complexing, condensing to a liquid, condensing to a solid, adjusting the pH, converting from a liquid to a gas, converting from a solid to a liquid or gas, change the chemical state, change the physical state, or any combination thereof. In some embodiment [2310] and [2312] are press fit into at least one of the cap or body. In some embodiments, internal structures are incorporated into the cap [2305] or the body [2306] to prevent any of [2310], [2311], and/or [2312] from moving within the capsule. Multiple layers and combinations of filters, membranes, frits and materials for conditioning the gas stream are possible as described in previous figures. In some embodiments, the cap [2305] has a length of 12.95 mm, and an external diameter of 9.91 mm. In other embodiments, the cap [2305] has a length of 11.74 mm, and an external diameter of 8.53 mm. In other embodiments, the cap [2305] has a length of 10.72 mm, and an external diameter of 7.64 mm. In other embodiments, the cap [2305] has a length of 9.78 mm, and an external diameter of 6.91 mm. In other embodiments, the cap [2305] has a length of 8.94 mm, and an external diameter of 6.35 mm. In other embodiments, the cap [2305] has a length of 8.08 mm, and an external diameter of 5.82 mm. In other embodiments, the cap [2305] has a length of 7.21 mm, and an external diameter of 5.32 mm. In some embodiments the cap [2305] has a length less than 20 mm, and an external diameter less than 20 mm. In some embodiments the body [2306] has a length of 22.2 mm, and an external diameter of 9.55 mm. In some embodiments the body [2306] has a length of 20.2 mm, and an external diameter of 8.18 mm. In some embodiments the body [2306] has a length of 18.44 mm, and an external diameter of 7.34 mm. In some embodiments the body [2306] has a length of 16.61 mm, and an external diameter of 6.63 mm. In some embodiments the body [2306] has a length of 15.27 mm, and an external diameter of 6.07 mm. In some embodiments the body [2306] has a length of 13.59 mm, and an external diameter of 5.57 mm. In some embodiments the body [2306] has a length of 12.19 mm, and an external diameter of 5.05 mm. In some embodiments the body [2306] has a length less than 25 mm, and an external diameter less than 25 mm. In some embodiments, the capsule [2301] has an internal volume capacity of 1370 ul, and an overall closed length of 26.1 mm. In some embodiments, the capsule [2301] has an internal volume capacity of 910 ul, and an overall closed length of 23.3 mm. In some embodiments, the capsule [2301] has an internal volume capacity of 680 ul, and an overall closed length of 21.7 mm. In some embodiments, the capsule [2301] has an internal volume capacity of 500 ul, and an overall closed length of 19.4 mm. In some embodiments, the capsule [2301] has an internal volume capacity of 370 ul, and an overall closed length of 18.0 mm. In some embodiments, the capsule [2301] has an internal volume capacity of 300 ul, and an overall closed length of 15.9 mm. In some embodiments, the capsule [2301] has an internal volume capacity of 210 ul, and an overall closed length of 14.3 mm. In some embodiments, the capsule [2301] has an internal volume capacity less than 2000 ul, and an overall closed length of less than 50 mm. In some embodiments the dimensions and volume of the cap [2304], the body [2306] and the capsule [2301] may differ from those listed here.

Cartridge or capsule dimensions may be selected to match standard sizes associated with pharmaceutical capsules to facilitate high volume production. Examples include:

| | Embodiment Number | | | | | | |
|---|---|---|---|---|---|---|---|
| | 1 | 2 | 3 | 4 | 5 | 6 | 7 |
| | Capsule Standard Size | | | | | | |
| | 0000 | 00 | 0 | 1 | 2 | 3 | 4 |
| Internal Capsule | | | | | | | |
| Volume | | | | | | | |
| Volume in ml | 1.37 | 0.91 | 0.68 | 0.5 | 0.37 | 0.3 | 0.21 |
| Length | | | | | | | |
| Body millimeters | 22.2 | 20.22 | 18.44 | 16.61 | 15.27 | 13.59 | 12.19 |
| Cap millimeters | 12.95 | 11.74 | 10.72 | 9.78 | 8.94 | 8.08 | 7.21 |
| External diameter | | | | | | | |
| Body millimeters | 9.55 | 8.18 | 7.34 | 6.63 | 6.07 | 5.57 | 5.05 |
| Cap millimeters | 9.91 | 8.53 | 7.64 | 6.91 | 6.35 | 5.82 | 5.32 |
| Overall closed length | | | | | | | |
| Millimeters | 26.1 | 23.3 | 21.7 | 19.4 | 18 | 15.9 | 14.3 |

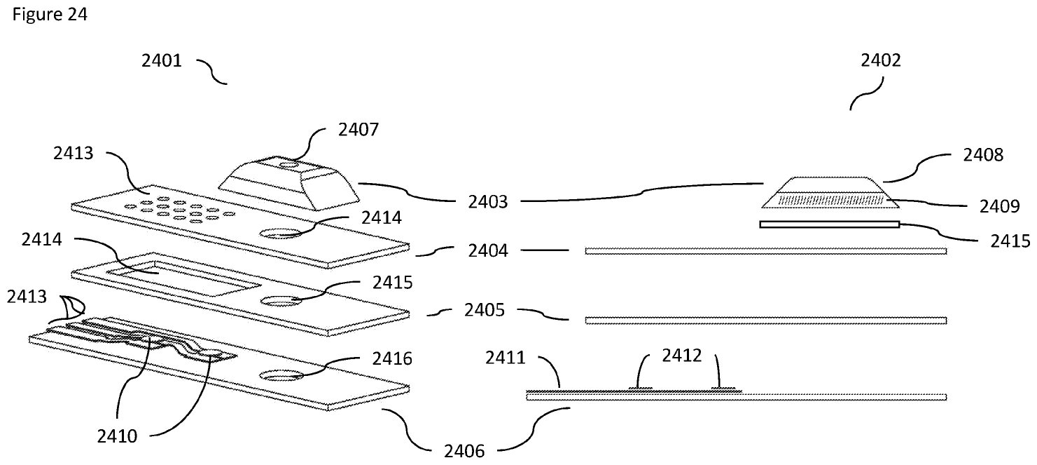

FIG. 24 depicts an exploded perspective [2401] and side view [2402] of an embodiment of an integrated gas conditioning test strip. The test strip comprising of a chamber [2403], a protective layer [2404], a spacing layer [2405] and a sensing layer [2406]. The chamber [2403] further defines at least one inlet hole [2407], at least one outlet hole (not shown), and comprises at least one of a membrane, filter, or frit [2408], at least one of a material to condition the gas [2409], and at least one of a second membrane, filter, or frit [2415] to encapsulate the material [2409]. The membrane, filter, or frits [2408] and [2415] may be internal or external to the chamber. The protective spacing layers [2404] and [2405] are further define at least one hole [2414] and [2415]. The sensing layer [2406] further defines at least one hole [2416]. The sensing layer [2406] is further comprised of at least one electrode [2413, 2411] and at least one sensing chemistry [2410, 2412]. The at least one hole in the layers [2404, 2405, 2406] is configured to enable fluid communication between the chamber inlet [2707] and the additional layers [2404, 2405, 2406]. In one embodiment, the protective [2404] and spacing layers [2405] further define at least one of a second hole [2413 and 2414]. The at least one second hole in the layers is configured to enable fluid communication with a sensor (if a sensing layer is not present) or sensing chemistry [2410, 2412] on the sensing layer [2406] if present.

In a preferred embodiment, the flow of the conditioned and unconditioned gas through the sensor is described in FIGS. 18 and 19 and 25. In some embodiments, the layer [2412] is not present. In some embodiments the sensing layer [2406] is not present.

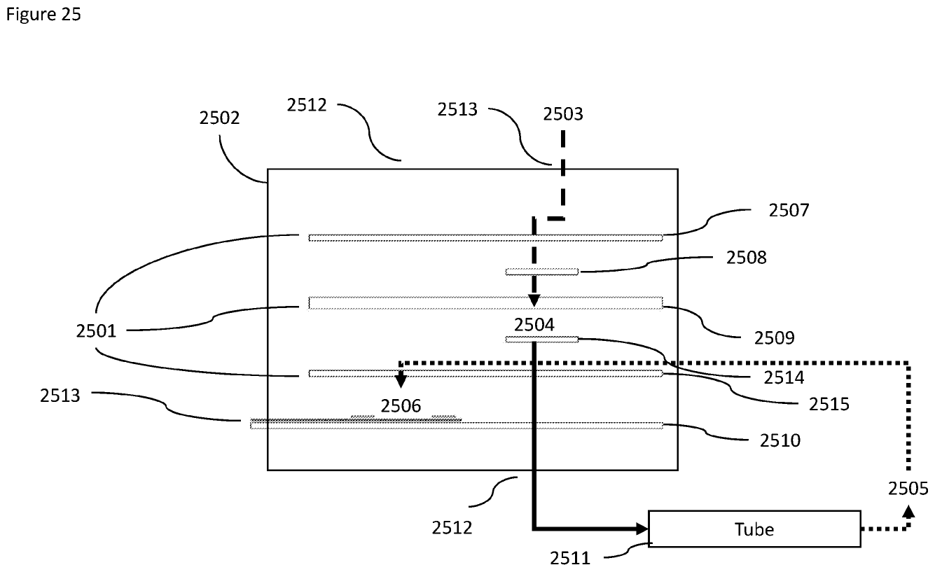

FIG. 25 depicts a preferred embodiment of a gas conditioning system. The embodiment comprises at least a first protective layer [2507], at least one first membrane layer [2508], at least one first spacing layer [2509] further defining at least one hole in which at least one material to condition the gas is disposed, at least one second membrane layer [2514], at least one second spacing layer [2515] and a sensing layer [2506] further comprising at least one electrode [2513] and at least one sensing chemistry [2506]. The at least first protective layer [2507], second spacing layer [2515] and sensing layer [2506] is further define at least one hole. In one embodiment, the second spacing layer [2515] is not present. In one embodiment, the second spacing layer [2515] and sensing layer [2510] is not present. In one embodiment, the sensing layer [2510] is not present.

In this embodiment, a test strip ([2501] combined with a sensing layer [2513]) is inserted into a device chamber [2502] as previously described. The unconditioned gas [2503] enters into the device chamber [2513] and the at least one first protective layer of the test strip [2507], it passes through the at least one first membrane layer [2508] and into the at least one spacing layer containing material to condition the gas stream [2509] through the holes defined therein as previously described. The conditioned gas [2504] passes through the at least one second membrane layer [2514], at least one second spacing layer [2515] and a sensing layer [2510] through the holes defined therein, exits the device chamber [2512] and enters the tube [2511] where it is conditioned a second time. The twice conditioned gas [2505] enters the device chamber [2512] a second time and is passed through the layers [2501] to the at least one sensing chemistry [2506] for analysis.

In one embodiment, the material in the first spacing layer [2509] is one of a silica, permanganate salt or a permanganate sale on silica and the tube comprises one or more of a perfluorosulfonic acid or a polymer or copolymer derived therefrom, a perflurocarboxylic acid or a polymer or copolymer derived therefrom, or a humidity exchange material.

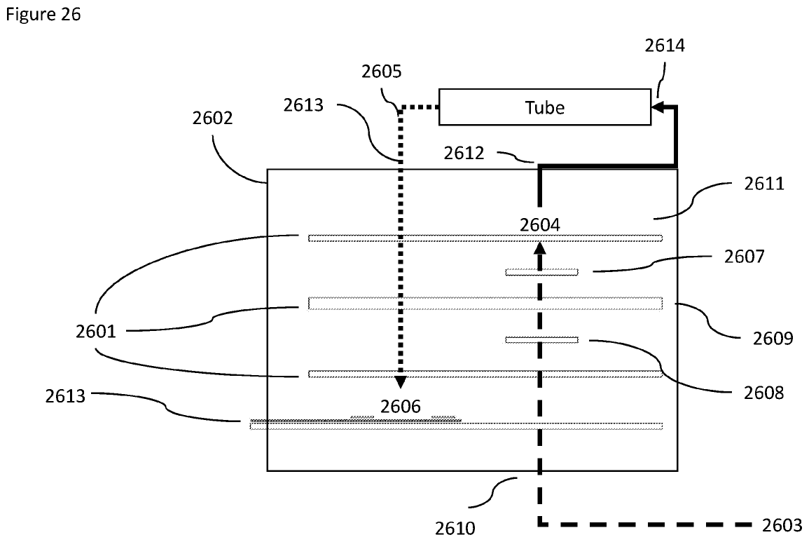

FIG. 26 depicts a preferred embodiment of a gas conditioning system. It is analogous to FIG. 25 except the gas flows into the opposite end of the test strip. A test strip ([2601] combined with a sensing layer [2613]) is inserted into a device chamber [2602] as previously described. The unconditioned gas [2603] enters into the device chamber [2610] and through a hole in the sensing layer [2613] it passes through the first membrane layer [2608] and into the spacing layer containing material to condition the gas stream [2609] as previously described. In a preferred embodiment, the material contains at least one of a permanganate salt, a permanganate salt on silica. The conditioned gas [2604] passes through the remaining layers [2607] and [2611], exits the device chamber [2612] and enters the tube [2614] where it is conditioned a second time. The twice conditioned gas [2605] enters the device chamber [2513] a second time and is passed through the layers [2601] to the at least one sensing chemistry [2606] for analysis.

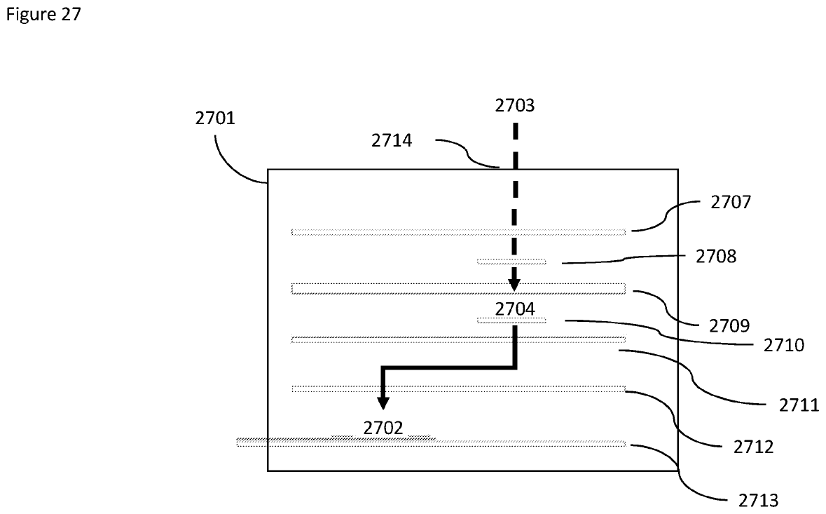

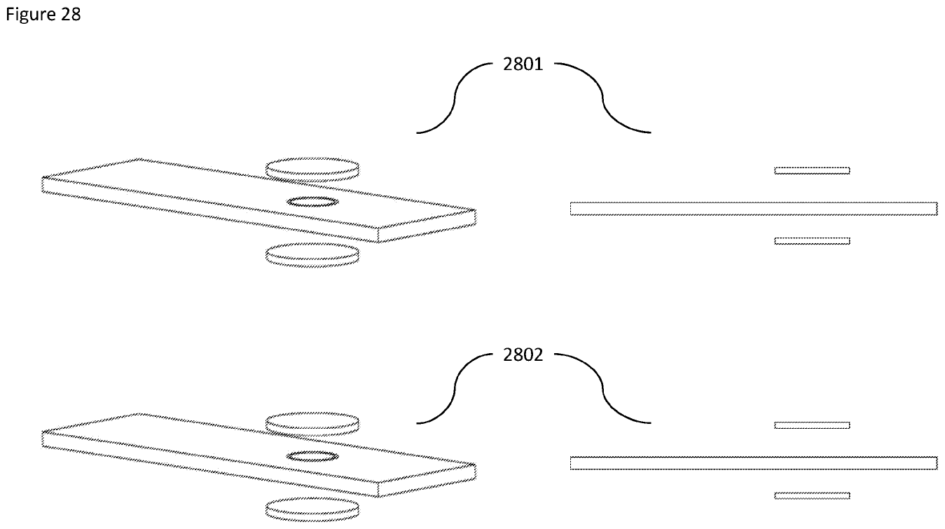

FIG. 27 depicts one embodiment of a gas conditioning system. A test strip is placed inside a device chamber [2701] the test trip comprising of a protective layer [2707], a first membrane layer [2708], a first spacing layer [2709], a second membrane layer [2710], a second spacing layer [2711], a third spacing layer [2712], and a sensing layer [2706]. The first spacing layer [2709] further defines at least one hole through the layer wherein a material to condition the gas stream is disposed in the hole. Suitable materials have been previously described. In a preferred embodiment, the material contains at least one of a permanganate salt, a permanganate salt on silica. In a preferred embodiment, the permanganate salt is potassium. The first protective layer [2707], second spacing layer [2711] further comprising of at least one hole and the third spacing layer [2712] comprising a channel in fluid communication with the sensing chemistry. The test strip configured such that the at least one hole in the layers [2707], [2709], [2711] and the channel in layer [2712] are in fluid communication such that the gas sample [2703] provided by the device (not shown) may pass into the chamber [2714] through the protective layer [2707], first membrane layer [2708], first spacing layer [2709], second membrane layer [2710], second spacing layer [2711], and third spacing layer [2712], to the sensing chemistry [2702] located on the sensing layer [2713]. In one embodiment, the second spacing layer [2711] is not present.

Aspects of the techniques and systems related to measuring the concentration of an analyte in a fluid sample and/or performing a calibration on the devices as disclosed herein may be implemented as a computer program product for use with a computer system or computerized electronic device, using, e.g., a processor/microprocessor. Such implementations may include a series of computer instructions, or logic, fixed either on a tangible/non-transitory medium, such as a computer readable medium (e.g., a diskette, CDROM, ROM, flash memory or other memory or fixed disk) or transmittable to a computer system or a device, via a modem or other interface device, such as a communications adapter connected to a network over a medium.

The medium may be either a tangible medium (e.g., optical or analog communications lines) or a medium implemented with wireless techniques (e.g., Wi-Fi, cellular, microwave, infrared or other transmission techniques). The series of computer instructions embodies at least part of the functionality described herein with respect to the system. Those skilled in the art should appreciate that such computer instructions can be written in a number of programming languages for use with many computer architectures or operating systems.

Such instructions may be stored in any tangible memory device, such as semiconductor, magnetic, optical or other memory devices, and may be transmitted using any communications technology, such as optical, infrared, microwave, or other transmission technologies.

It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein.

1. A system comprising:
a test strip comprising:
one or more flexible layers defining one or more flexible layer holes, and
one or more of a permanganate salt, silica, permanganate salt on silica, or activated carbon disposed in the one or more flexible layer holes; and
a tube in fluid communication with the test strip, wherein the tube comprises one or more of a perfluorosulfonic acid or a polymer or copolymer derived therefrom, a perflurocarboxylic acid or a polymer or copolymer derived therefrom, or a humidity exchange material; and
one or more sensors to detect and/or measure an analyte.

2. The system of claim 1, wherein the permanganate salt on silica is deposited in the one or more flexible layer holes.

3. The system of claim 2, wherein the permanganate salt on silica is a potassium permanganate.

4. The system of any of claims 1 to 3, wherein the one or more flexible layer holes is tapered.

5. The system of any of claims 1 to 4, wherein the one or more flexible layer holes is circular, oval-shaped, square-shaped, or rectangular.

6. The system of any of claims 1 to 5 further comprising one or more membrane layers.

7. The system of claim 6, wherein the one or more membrane layers comprise
a first membrane layer, and
a second membrane layer;
wherein the one or more flexible layers comprises a first flexible layer,
wherein the first flexible layer has a first upper surface, wherein the first flexible layer has a first lower surface, and wherein the first flexible layer defines a first hole traversing the first upper surface and the first lower surface,
wherein the first membrane is configured to overlay the first hole defined by the first upper surface of the first flexible layer, and
wherein the second membrane layer has a first second-membrane surface, wherein the second membrane layer has a second second-membrane surface, and wherein the first second-membrane surface is configured to overlay the first hole defined by the first lower surface of the first flexible layer.

8. The system of claim 7, wherein the one or more of the permanganate salt, the silica, the permanganate salt on silica, or the activated carbon is deposited in the first hole.

9. The system of claim 8, wherein the permanganate salt on silica is deposited in the first hole.

10. The system of claim 9, wherein the permanganate salt on silica is a potassium permanganate.

11. The system of any of claims 7 to 10,
wherein the one or more flexible layers further comprises a second flexible layer,
wherein the one or more membrane layers further comprises a third membrane layer,
wherein the second flexible layer has a second upper surface, wherein the second flexible layer has a second lower surface, and wherein the second flexible layer defines a second hole traversing the second upper surface and the second lower surface,
wherein the second membrane layer is disposed between the first flexible layer and the second flexible layer, and
wherein the third membrane layer is configured to overlay the second hole defined by the second lower surface of the second flexible layer.

12. The system of claim 11,
wherein the one or more membrane layers further comprises a fourth membrane layer,
wherein the fourth membrane layer has a first fourth-membrane surface, wherein the fourth membrane layer has a second fourth-membrane surface, wherein the fourth membrane is disposed between the second membrane layer and the second flexible layer, and wherein the second fourth-membrane surface is configured to overlay the second hole defined by the second upper surface of the second flexible layer.

13. The system of claim 6,
wherein the total number of the one or more flexible layers is n,
wherein the total number of the one or more membranes is m, and
wherein m is equal to n, n+1, or n−1.

14. The system of claim 11 or claim 12, wherein the one or more of the permanganate salt, the silica, the permanganate salt on silica, or the activated carbon is deposited in the second hole.

15. The system of claim 14, wherein the permanganate salt on silica is deposited in the second hole.

16. The system of claim 15, wherein the permanganate salt on silica deposited in the second hole is a potassium permanganate.

17. The system of any of claims 7 to 16 further comprising one or more protective layers, wherein the one or more protective layers comprises a first protective layer configured to overlay the second surface of the first membrane layer.

18. The system of claim 17, wherein the first protective layer defines a protective layer hole.

19. The system of claim 18, wherein the protective layer hole defined by the first protective layer is configured to provide fluid communication between the one or more of the permanganate salt, the silica, the permanganate salt on silica, or the activated carbon of the test strip and the tube.

20. The system of any of claims 1 to 19, wherein the sensor is a sensing layer.

21. The system of claim 20, wherein the test strip comprises the sensing layer.

22. The system of claim 20 or claim 21, wherein the sensing layer defines one or more sensing layer holes.

23. The system of claim 22, wherein the one or more sensing layer holes defined by the sensing layer is configured to provide fluid communication between the one or more of the permanganate salt, the silica, the permanganate salt on silica, or the activated carbon of the test strip and the tube.

24. The system of any of claims 21 to 23, wherein the sensing layer comprises one or more electrodes.

25. The system of any of claims 21 to 23, wherein the sensing layer comprises one or more sensing chemistries.

26. The system of claim 25,
wherein the sensing layer further comprises one or more electrodes, and
wherein the one or more sensing chemistries is configured to bridge the one or more electrodes.

27. The system of any of claims 21 to 26,
wherein the test strip comprises one or more spacing layers, and
wherein the one or more spacing layers defines one or more spacing layer holes.

28. The system of any of claims 1 to 27,
wherein the system further comprises a housing, and
wherein the housing is configured to provide fluid communication between one or more of the test strip, the one or more sensors, and the tube.

29. The system of claim 28, wherein the housing is configured to provide fluid communication between the test strip and the tube.

30. The system of claim 28 or claim 29 further comprising a pump, a blower, or a fan connected to the housing, wherein the pump, the blower, or the fan is configured advance a gas through the system.

31. A system comprising
a test strip comprising:
one or more flexible layers defining one or more flexible layer holes,
one or more of a permanganate salt, silica, permanganate salt on silica, or activated carbon disposed in the one or more flexible layer holes, and
one or more spacing layers defining one or more channels; and
one or more sensors to detect and/or measure an analyte,
wherein the one or more channels are configured to provide fluid communication for a gas between the test strip and the one or more sensors.

32. The system of claim 31, wherein the one or more channels provide fluid communication for the gas to the one or more sensors subsequent to the gas traversing the one or more of the permanganate salt, the silica, the permanganate salt on silica, or the activated carbon of the test strip.

33. The system of claim 31 or claim 32, wherein the permanganate salt on silica is deposited in the one or more flexible layer holes.

34. The system of claim 33, wherein the permanganate salt on silica is a potassium permanganate.

35. The system of any of claims 31 to 34, wherein the one or more flexible layer holes is tapered.

36. The system of any of claims 31 to 35, wherein the one or more flexible layer holes is circular, oval-shaped, square-shaped, or rectangular.

37. The system of any of claims 31 to 36 further comprising one or more membrane layers.

38. The system of claim 37, wherein the one or more membrane layers comprise
a first membrane layer, and
a second membrane layer;
wherein the one or more flexible layers comprises a first flexible layer,
wherein the first flexible layer has a first upper surface, wherein the first flexible layer has a first lower surface, and wherein the first flexible layer defines a first hole traversing the first upper surface and the first lower surface,
wherein the first membrane is configured to overlay the first hole defined by the first upper surface of the first flexible layer, and
wherein the second membrane layer has a first second-membrane surface, wherein the second membrane layer has a second second-membrane surface, and wherein the first second-membrane surface is configured to overlay the first hole defined by the first lower surface of the first flexible layer.

39. The system of claim 38, wherein the one or more of the permanganate salt, the silica, the permanganate salt on silica, or the activated carbon is deposited in the first hole.

40. The system of claim 39, wherein the permanganate salt on silica is deposited in the first hole.

41. The system of claim 40, wherein the permanganate salt on silica is a potassium permanganate.

42. The system of any of claims 38 to 41,
wherein the one or more flexible layers further comprises a second flexible layer,
wherein the one or more membrane layers further comprises a third membrane layer,
wherein the second flexible layer has a second upper surface, wherein the second flexible layer has a second lower surface, and wherein the second flexible layer defines a second hole traversing the second upper surface and the second lower surface,
wherein the second membrane layer is disposed between the first flexible layer and the second flexible layer, and
wherein the third membrane layer is configured to overlay the second hole defined by the second lower surface of the second flexible layer.

43. The system of claim 42,
wherein the one or more membrane layers further comprises a fourth membrane layer,
wherein the fourth membrane layer has a first fourth-membrane surface, wherein the fourth membrane layer has a second fourth-membrane surface, wherein the fourth membrane is disposed between the second membrane layer and the second flexible layer, and wherein the second fourth-membrane surface is configured to overlay the second hole defined by the second upper surface of the second flexible layer.

44. The system of claim 37,
wherein the total number of the one or more flexible layers is n,
wherein the total number of the one or more membranes is m, and
wherein m is equal to n+1.

45. The system of claim 42 or claim 43, wherein the one or more of the permanganate salt, the silica, the permanganate salt on silica, or the activated carbon is deposited in the second hole.

46. The system of claim 45, wherein the permanganate salt on silica is deposited in the second hole.

47. The system of claim 46, wherein the permanganate salt on silica is a potassium permanganate.

48. The system of any of claims 38 to 47 further comprising one or more protective layers, wherein the one or more protective layers comprises a first protective layer configured to overlay the second first-membrane surface of the first membrane layer.

49. The system of claim 48, wherein the first protective layer defines a protective layer hole.

50. The system of any of claims 31 to 49, wherein the sensor is a sensing layer.

51. The system of claim 50, wherein the test strip comprises the sensing layer.

52. The system of claim 50 or claim 51, wherein the sensing layer defines one or more sensing layer holes.

53. The system of claim 51 or claim 52, wherein the sensing layer comprises one or more electrodes.

54. The system of claim 51 or claim 52, wherein the sensing layer comprises one or more sensing chemistries.

55. The system of claim 54,
wherein the sensing layer further comprises one or more electrodes, and
wherein the one or more sensing chemistries is configured to bridge the one or more electrodes.

56. The system of any of claims 1 to 55 further comprising one or more chamber layers at least in part defining a chamber, and
wherein the chamber comprises one or more of a chamber membrane, a chamber frit, or a chamber filter.

57. The system of claim 56, wherein the one or chamber layers comprises
one or more protective layers, and/or
one or spacing layers.

58. The system of claim 56 or claim 57, wherein the chamber comprises one or more of a permanganate salt, silica, a permanganate salt on silica, or an activated carbon.

59. The system of claim 58, wherein the chamber comprises the permanganate salt on silica.

60. The system of any of claims 56 to 59, wherein the chamber is tapered.

61. The system of any of claims 1 to 60 further comprising one or more of:
a pressure sensitive adhesive;
a heat sensitive adhesive;
a sonic weld;
a bond;
a two-part adhesive; or
a moisture-cure adhesive.

62. The system of any of claims 1 to 61 further comprising one or more humectants.

63. The system of claim 62, wherein the one or more humectants comprises:
polypropylene glycol;
glycerin;
sodium hexamethyl phosphate;
a glycol;
a sugar alcohol; or
glyceryl triacetate.

64. The system of any of claims 1 to 63 further comprising one or more desiccants.

65. The system of claim 64 wherein the one or more desiccants comprises:
a silica gel;
an activated alumina;
a bentonite clay;
calcium sulfate;
magnesium sulfate; or
sodium chloride.

66. The system of any of claims 1 to 65 further comprising one or more humidity stabilizing materials.

67. The system of claim 66, wherein the one or more humidity stabilizing materials comprises:
magnesium chloride;
a hydroxylmethyl cellulose composites;
a clay composite;
a silica gel; or
Propadyn

68. The system of any of claims 1 to 67, wherein the one or more sensors comprises a chemoreceptive sensor.

69. The system of any of claims 1 to 67, wherein the one or more sensors comprises a metal oxide sensor.

70. The system of any of claims 1 to 67, wherein the one or more sensors comprises a electrochemical sensor.

71. The system of any of claims 1 to 67, wherein the one or more sensors comprises a chemiresistive sensor.

72. A method of conditioning a gas sample, the gas sample having a humidity and comprising one or more input analytes, wherein the method comprises:
a. providing the gas sample to a gas sample receiver;
b. adjusting the humidity of the gas sample;
c. providing the gas sample to a tube comprising one or more of a perfluorosulfonic acid, a perflurocarboxylic acid, or a humidity exchange material; and
d. adjusting the humidity of the gas sample to conditions equal to or about equal to ambient humidity; and
e. detecting or measuring one or more readout analytes, wherein detecting or measuring the one or more readout analytes follows step (a) and step (b).

73. The method of claim 72,
wherein the gas sample receiver comprises one of a cartridge or a capsule,
wherein the cartridge or the capsule comprises one or more of one or more membranes, one or more frits, or one or more filters, and
wherein the gas sample passes through the one or more of the one or more membranes, the one or more frits, or the one or more filters in step (a).

74. The method of claim 73, wherein the one or more membranes, one or more fits, or one or more filters comprises one or more of a humidity exchange material, a selective membrane, a size exclusion membrane, a particulate filter, or a porous polypropylene.

75. The method of claim 72,
wherein the gas sample receiver comprises a test strip,
wherein the test strip comprises one or more of membranes, and
wherein the gas sample passes through the one or more membranes in step (a).

76. The method of claim 75, wherein the one or more membranes comprises one or more of a humidity exchange material, a selective membrane, a size-exclusion membrane, a particulate filter, or a porous polypropylene.

77. The method of claim 73 or claim 74,
wherein the cartridge or the capsule comprises one or more conditioning materials, and
wherein the gas sample passes through the one or more conditioning materials in step (a).

78. The method of claim 77, wherein the cartridge or the capsule comprises one or more humectants, and wherein the gas sample passes through the one or more humectants in step (a).

79. The method of claim 77 or claim 78, wherein the cartridge or the capsule comprises one or more desiccants, and wherein the gas sample passes through the one or more desiccants in step (a).

80. The method of any of claims 77 to 79, wherein the cartridge or the capsule comprises one or more humidity stabilizing materials.

81. The method of claim 75 or claim 76,
wherein the test strip comprises one or more conditioning materials, and
wherein the gas sample passes through the one or more conditioning materials in step (a).

82. The method of claim 81, wherein the test strip comprises one or more humectants, and
wherein the gas sample passes through the one or more humectants in step (a).

83. The method of claim 81 or claim 82, wherein the test strip comprises one or more desiccants, and wherein the gas sample passes through the one or more desiccants in step (a).

84. The method of any of claims 81 to 83, wherein the cartridge or the capsule comprises one or more humidity stabilizing materials.

85. The method of any of claim 78, 79, 80, 82, 83, or 84 wherein the one or more humectants comprises:
polypropylene glycol;
glycerin;
sodium hexamethyl phosphate;
a glycol;
a sugar alcohol; or
glyceryl triacetate.

86. The method of any of the claim 79, 80, 83, or 84 wherein the one or more desiccants comprises:
a silica gel;
an activated alumina;
a bentonite clay;
calcium sulfate;
magnesium sulfate; or
sodium chloride.

87. The method of claim 80 or claim 84 wherein the one or more humidity stabilizing materials comprises:
magnesium chloride;
a hydroxylmethyl cellulose composites;
a clay composite;
a silica gel; or
Propadyn.

88. The method of any of claims 77 to 87, wherein the adjusting the humidity of the gas sample in step (b) is a result of the gas sample passing through the one or more conditioning materials.

89. The method of any of claims 77 to 88, wherein the one or more conditioning materials comprises one or more of permanganate salt, silica, permanganate salt on silica, or activated carbon.

90. The method of claim 89, wherein the one or more conditioning materials comprises permanganate salt on silica.

91. The method of claim 90, wherein the permanganate salt on silica is a potassium permanganate on silica.

92. The method of any of claims 89 to 91, wherein step (a) and step (b) occur substantially simultaneously.

93. The method of any of claims 72 to 92, wherein the adjusting the humidity of the gas sample in step (b) decreases the humidity of the gas sample.

94. The method of any of claims 72 to 92, wherein the adjusting the humidity of the gas sample in step (b) increases the humidity of the gas sample.

95. The method of any of claims 72 to 94, wherein the gas sample passes through the tube in step (c).

96. The method of claim 95, wherein the adjusting the humidity of the gas sample to conditions equal to or about equal to ambient humidity in step (d) is a result of passing through the tube.

97. The method of any of claims 72 to 96, wherein the one or more input analytes comprises a first input analyte, and wherein the one or more readout analytes comprises a first readout analyte, the method further comprising:
f. before step (e), altering the first input analyte chemically, thereby providing the first readout analyte.

98. The method of claim 97, wherein step (f) comprises oxidizing the first input analyte.

99. The method of claim 97, wherein step (f) comprises reducing the first input analyte.

100. The method of claim 97, wherein step (f) comprises sorbing one or more contaminants.

101. The method of claim 97,
wherein the gas sample has a pH level, and
wherein step (f) comprises adjusting the pH level of the gas sample.

102. The method of claim 97,
wherein the gas sample has an ionic charge, and
wherein step (f) comprises adjusting the ionic charge of the gas sample.

103. The method of claim 97, wherein step (f) comprises one or more of oxidizing the first input analyte, reducing the first input analyte, sorbing one or more contaminants, adjusting a pH level of the gas sample, or adjusting an ionic charge of the gas sample.

104. The method of any of claims 97 to 103,
wherein step (f) follows step (a) and step (b), and
wherein step (f) precedes step (c), step (d), and step (e).

105. The method of any of claims 97 to 103,
wherein step (f) follows step (a), and
wherein step (f) precedes step (b), step (c), step (d), and step (e).

106. The method of any of claims 97 to 103,
wherein step (c) and step (d) precede step (a) and step (b).

107. The method of any of claims 97 to 103, wherein step (f) immediately precedes step (b).

108. The method of any of claims 97 to 103, wherein step (b) immediately precedes step (f).

109. The method of any of claims 97 to 103, wherein step (b) and step (f) occur substantially simultaneously.

110. The method of any of claims 97 to 109, wherein the gas sample is a breath sample from a human or an animal.

111. The method of any of claims 97 to 109, wherein the gas sample is provided by a pump, a diffusion, or a vacuum.

112. The method of claim 110 or claim 111, wherein the first input analyte is nitric oxide.

113. The method of claim 112, wherein the first readout analyte is nitrogen dioxide.

114. The method of claim 113, wherein the concentration of nitric oxide in the breath sample is determined using the detection or measurement of nitrogen dioxide in step (e).

115. The method of any of claims 72 to 96,
wherein the one or more input analytes comprises a first input analyte,
wherein the one or more readout analyte comprises a first readout analyte, and
wherein the first input analyte is the same as the first readout analyte.

116. The method of claim 115, wherein the gas sample is a breath sample from a human or an animal.

117. The method of claim 115, wherein the gas sample is provided by a pump, a diffusion, or a vacuum.

118. The method of any of claims 115 to 117, wherein the first input analyte comprises nitric oxide.

119. The method of any of claims 72 to 118, wherein the detecting or measuring one or more readout analytes is performed by a chemoreceptive sensor.

120. The method of any of claims 72 to 118, wherein the detecting or measuring one or more readout analytes is performed by a metal oxide sensor.

121. The method of any of claims 72 to 118, wherein the detecting or measuring one or more readout analytes is performed by a electrochemical sensor.

122. The method of any of claims 72 to 118, wherein the detecting or measuring one or more readout analytes is performed by a chemiresistive sensor.

123. A system comprising
an enclosure comprising:
one or more of a frit, a filter, or a membrane, and
one or more of a permanganate salt, silica, permanganate salt on silica, or activated carbon; and
a tube in fluid communication with the enclosure, wherein the tube comprises one or more of a perfluorosulfonic acid or a polymer or copolymer derived therefrom,
a perflurocarboxylic acid or a polymer or copolymer derived therefrom, or a humidity exchange material; and
one or more sensors to detect and/or measure an analyte;
wherein the enclosure is a cartridge or a capsule.

124. The system of claim 123, wherein the enclosure defines an inlet.

125. The system of claim 123 or claim 124, wherein the enclosure defines an outlet.

126. The system of any of claims 123 to 125,
wherein the one or more of a frit, a filter, or a membrane comprises a first frit, a first filter, or a first membrane,
wherein the one or more of a permanganate salt, silica, permanganate salt on silica, or activated carbon comprises a first permanganate salt, a first silica, a first permanganate salt on silica, or a first activated carbon,
wherein the one or more of a frit, a filter, or a membrane comprises a second frit, a second filter, or a second membrane,
wherein the first permanganate salt, the first silica, the first permanganate salt on silica, or the first activated carbon is disposed between
the first frit, the first filter, or the first membrane; and
the second frit, the second filter, or the second membrane.

127. The system of any of claims 123 to 126, wherein the one or more of a frit, a filter, or a membrane define one or more pores.

128. The system of claim 127,
wherein the one or more of the permanganate salt, silica, permanganate salt on silica, or activated carbon has a particle size, and
wherein the one or more pores is less than the particle size of the one or more of the potassium permanganate, silica, potassium permanganate on silica, or activated carbon.

129. The system of claim 127 or claim 128, wherein the one or more pores have one or more pore sizes are configured to permit a gas sample passage to traverse the one or more of frit, a filter, or a membrane.

130. The system of any of claims 123 to 129,
wherein the system further comprises a housing, and
wherein the housing is configured to provide fluid communication between the enclosure and the tube.

131. The system of claim 130, wherein the housing is configured to further provide fluid communication between
the enclosure and the tube, and
the one or more sensors.

132. The system of claim 130 or claim 131 further comprising a pump, a blower, or a fan connected to the housing, wherein the pump, the blower, or the fan is configured advance a gas through the system.

133. The system of any of the claims 123 to 132,
wherein the enclosure is a capsule,
wherein the capsule comprises a cap section and a body section, and
wherein the cap section and the body section are configured to press fit together.

134. The system of claim 133, wherein the cap section defines one or more cap holes.

135. The system of claim 133, wherein the body section defines one or more body holes.

136. The system of claim 134, wherein the body section defines one or more body holes.

137. The system of claim 134 or claim 136,
wherein the one or more cap holes comprises a first cap hole, and
wherein the cap section and the body section are press fit together, thereby covering the first cap hole.

138. The system of claim 135 or claim 136,
wherein the one or more body holes comprises a first body hole, and
wherein the cap section and the body section are press fit together, thereby covering the first body hole.

139. The system of any of claims 123 to 138 further comprising one or more of:
a pressure sensitive adhesive;
a heat sensitive adhesive;
a sonic weld;
a bond;
a two-part adhesive; or
a moisture-cure adhesive.

140. The system of any of claims 123 to 139 further comprising one or more humectants.

141. The system of claim 140, wherein the one or more humectants comprises:
polypropylene glycol;
glycerin;
sodium hexamethyl phosphate;
a glycol;
a sugar alcohol; or
glyceryl triacetate.

142. The system of any of claims 123 to 141 further comprising one or more desiccants.

143. The system of claim 142 wherein the one or more desiccants comprises:
a silica gel;
an activated alumina;
a bentonite clay;
calcium sulfate;
magnesium sulfate; or
sodium chloride.

144. The system of any of claims 123 to 143 further comprising one or more humidity stabilizing materials.

145. The system of claim 144, wherein the one or more humidity stabilizing materials comprises:
magnesium chloride;
a hydroxylmethyl cellulose composites;
a clay composite;
a silica gel; or
Propadyn.

146. The system of claims 123 to 145, wherein the one or more sensors comprises a chemoreceptive sensor.

147. The system of any of claims 123 to 145, wherein the one or more sensors comprises a metal oxide sensor.

148. The system of any of claims 123 to 145, wherein the one or more sensors comprises a electrochemical sensor.

149. The system of any of claims 123 to 145, wherein the one or more sensors comprises a chemiresistive sensor.

150. The system of any of claims 123 to 149, wherein the enclosure comprises the permanganate salt on silica.

151. The system of claim 150, wherein the permanganate salt on silica is a potassium permanganate.

152. A system comprising
an enclosure comprising:
one or more of a frit, a filter, or a membrane, and
one or more of a permanganate salt, silica, permanganate salt on silica, or activated carbon; and
one or more sensors to detect and/or measure an analyte;
wherein the enclosure is a cartridge or a capsule.

153. The system of claim 152, wherein the enclosure defines an inlet.

154. The system of claim 152 or claim 153, wherein the enclosure defines an outlet.

155. The system of any of claims 152 to 154,
wherein the one or more of a frit, a filter, or a membrane comprises a first frit, a first filter, or a first membrane,
wherein the one or more of a permanganate salt, silica, permanganate salt on silica, or activated carbon comprises a first permanganate salt, a first silica, a first permanganate salt on silica, or a first activated carbon,
wherein the one or more of a frit, a filter, or a membrane comprises a second frit, a second filter, or a second membrane,
wherein the first permanganate salt, the first silica, the first permanganate salt on silica, or the first activated carbon is disposed between
the first frit, the first filter, or the first membrane; and
the second frit, the second filter, or the second membrane.

156. The system of any of claims 152 to 155, wherein the one or more of a frit, a filter, or a membrane define one or more pores.

157. The system of claim 156,
wherein the one or more of the permanganate salt, silica, permanganate salt on silica, or activated carbon has a particle size, and
wherein the one or more pores is less than the particle size of the one or more of the potassium permanganate, silica, potassium permanganate on silica, or activated carbon.

158. The system of claim 156 or claim 157, wherein the one or more pores have one or more pore sizes are configured to permit a gas sample passage to traverse the one or more of frit, a filter, or a membrane.

159. The system of any of the claims 152 to 158,
wherein the enclosure is a capsule,
wherein the capsule comprises a cap section and a body section, and
wherein the cap section and the body section are configured to press fit together.

160. The system of claim 159, wherein the cap section defines one or more cap holes.

161. The system of claim 159, wherein the body section defines one or more body holes.

162. The system of claim 160, wherein the body section defines one or more body holes.

163. The system of claim 160 or claim 162,
wherein the one or more cap holes comprises a first cap hole, and
wherein the cap section and the body section are press fit together, thereby covering the first cap hole.

164. The system of claim 161 or claim 162,
wherein the one or more body holes comprises a first body hole, and
wherein the cap section and the body section are press fit together, thereby covering the first body hole.

165. The system of any of claims 152 to 164 further comprising one or more of:
a pressure sensitive adhesive;
a heat sensitive adhesive;
a sonic weld;
a bond;
a two-part adhesive; or
a moisture-cure adhesive.

166. The system of any of claims 152 to 165 further comprising one or more humectants.

167. The system of claim 166, wherein the one or more humectants comprises:
polypropylene glycol;
glycerin;
sodium hexamethyl phosphate;
a glycol;
a sugar alcohol; or
glyceryl triacetate.

168. The system of any of claims 152 to 167 further comprising one or more desiccants.

169. The system of claim 168 wherein the one or more desiccants comprises:
a silica gel;
an activated alumina;
a bentonite clay;
calcium sulfate;
magnesium sulfate; or
sodium chloride.

170. The system of any of claims 152 to 169 further comprising one or more humidity stabilizing materials.

171. The system of claim 170, wherein the one or more humidity stabilizing materials comprises:
magnesium chloride;
a hydroxylmethyl cellulose composites;
a clay composite;
a silica gel; or
Propadyn.

172. The system of claims 152 to 171, wherein the one or more sensors comprises a chemoreceptive sensor.

173. The system of any of claims 152 to 171, wherein the one or more sensors comprises a metal oxide sensor.

174. The system of any of claims 152 to 171, wherein the one or more sensors comprises a electrochemical sensor.

175. The system of any of claims 152 to 171, wherein the one or more sensors comprises a chemiresistive sensor.

176. The system of any of claims 152 to 171, wherein the enclosure comprises the permanganate salt on silica.

177. The system of claim 176, wherein the permanganate salt on silica is a potassium permanganate.
